(19) United States
(12) Patent Application Publication
(10) Pub. No.: US 2026/0283533 A1
(43) Pub. Date: Sep. 24, 2026

(54) SYSTEM

(71) Applicant: SoftBank Group Corp., Tokyo (JP)

(72) Inventor: Takuto TACHIMOTO, Tokyo (JP)

(73) Assignee: SoftBank Group Corp., Tokyo (JP)

(21) Appl. No.: 19/568,852

(22) Filed: Mar. 17, 2026

(30) Foreign Application Priority Data
Mar. 19, 2025 (JP) 2025-045253

(51) Int. Cl. A61B 5/372; A61B 5/00; G06N 3/09

(52) U.S. Cl. CPC A61B 5/372; A61B 5/7257; A61B 5/7267; G06N 3/09

(57) ABSTRACT

A system includes a processor that is configured to measure brain waves of a user and acquire the brain waves as digital signals, analyze the acquired digital signals to identify a thought content of the user, and generate a prompt that instructs a generative AI model to perform a conversion for converting the identified thought content into a natural language.

### CROSS-REFERENCE TO RELATED APPLICATION

This application is based on and claims priority under 35 USC 119 from Japanese Patent Application No. 2025-045253 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional human-computer interfaces require explicit physical or verbal actions, such as typing on a keyboard, touching a screen, or speaking into a microphone, in order to convey a user's intention. Users who have severe motor impairments or speech disabilities may not be able to perform such actions, which significantly restricts their ability to communicate with others and to operate digital devices. Even for users without impairments, existing interfaces often introduce latency and cognitive load because users must mentally formulate content and then manually or verbally express that content through a separate output action.

Moreover, traditional systems do not directly utilize brain activity as an input source to generate natural language, and therefore cannot seamlessly transform a user's internal thoughts into textual or spoken expressions. Existing brain-computer interface technologies, where available, tend to be limited in expressiveness, may rely on predefined commands, and lack integration with advanced generative AI models capable of producing fluent natural language in multiple languages. In addition, current solutions do not adequately support simultaneous multilingual conversations by dynamically translating a user's thought content into different languages through an automated pipeline.

Accordingly, there is a need for a system that directly acquires a user's brain waves, analyzes the corresponding digital signals to identify the user's thought content, and automatically converts such thought content into natural language by using a generative AI model. There is also a need for such a system to support multilingual communication, including simultaneous conversations in a plurality of languages, by instructing the generative AI model to perform translation between different languages based on the user's thought content.

### SUMMARY

To solve the above-described problems, an aspect of the present invention provides a system comprising a processor, wherein the processor is configured to measure brain waves of a user and acquire the brain waves as digital signals, analyze the acquired digital signals to identify a thought content of the user, and generate a prompt that instructs a generative AI model to perform a conversion for converting the identified thought content into a natural language. By directly linking the user's brain activity to natural language generation, the system enables the user's internal thoughts to be expressed as text or speech without requiring conventional physical or verbal input actions.

In one embodiment, the processor is further configured to generate the natural language by using the generative AI model with the prompt as an input. The processor may transmit the generated prompt to the generative AI model, obtain a natural language output corresponding to the identified thought content, and provide the output to a display device, a speaker, or another output interface. In this manner, the system can present the user's thought content as fluent, contextually appropriate sentences produced by the generative AI model.

In another embodiment, the processor is further configured to enable simultaneous conversation in a plurality of languages and to generate a prompt that instructs the generative AI model to perform translation between different languages. The processor may incorporate user-specific or application-specific language settings into the prompt so that the generative AI model outputs natural language expressions in one or more target languages. By doing so, the system can support real-time multilingual communication by converting a single identified thought content into a plurality of natural language outputs in different languages, thereby facilitating interaction between users who speak different languages and enhancing accessibility and communication efficiency.

The term “processor” refers to one or more hardware computing elements, such as a central processing unit (CPU), a graphics processing unit (GPU), a digital signal processor (DSP), an application-specific integrated circuit (ASIC), a field-programmable gate array (FPGA), or any combination thereof, capable of executing instructions to perform the functions described in the present specification and claims.

The term “brain waves” refers to electrical activity generated by neurons in a user's brain, which is detectable as analog electrical signals by an electroencephalogram (EEG) device or other brain activity sensor and is indicative of the user's cognitive or mental state.

The term “digital signals” refers to data obtained by converting analog electrical signals representing brain waves into discrete numerical values through an analog-to-digital conversion process, the data being suitable for storage, transmission, and processing by the processor.

The term “thought content” refers to semantic information, intent, meaning, or mental representation inferred from a user's brain waves, including but not limited to commands, statements, questions, emotional expressions, or conceptual ideas that the user internally conceives.

The term “analyze” refers to processing, transforming, or interpreting data using one or more algorithms, models, or computational procedures, including but not limited to filtering, feature extraction, classification, pattern recognition, or inference, to derive higher-level information from input signals.

The term “generative AI model” refers to a machine learning model, such as a neural network-based language model, configured to generate new data, including natural language text, in response to input data, prompts, or contextual information, and capable of producing coherent and contextually relevant output.

The term “prompt” refers to data, including textual or structured input, generated by the processor and provided to the generative AI model in order to specify, constrain, or guide the behavior of the generative AI model when generating natural language or performing translation.

The term “natural language” refers to a human language, such as English, Japanese, or any other spoken or written language, that is used for communication between humans and is characterized by grammar, vocabulary, and syntax understood by human users.

The term “translation” refers to a process of converting text or other linguistic content expressed in a source language into semantically corresponding text expressed in a different target language, while preserving the meaning or intent of the original content.

The term “simultaneous conversation in a plurality of languages” refers to a mode of operation in which the system outputs natural language expressions corresponding to a user's thought content in two or more different languages within overlapping or closely timed intervals, thereby enabling communication across multiple languages in real time or near real time.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

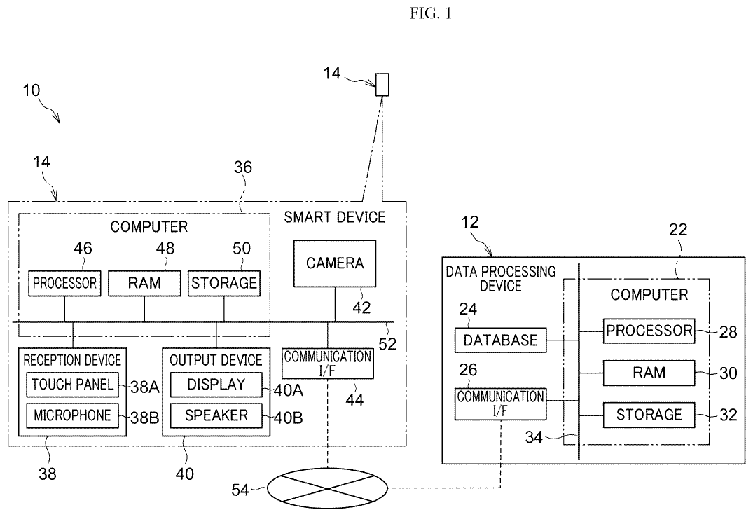

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

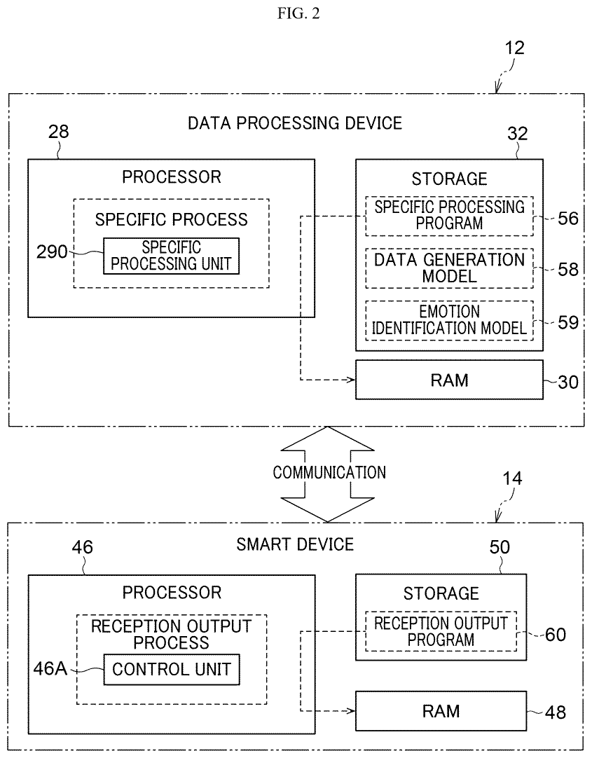

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional brain-signal based interaction systems suffer from multiple technical limitations in the way information is acquired, processed, and converted into output actions. First, existing systems often rely on simple thresholding or rule-based logic executed on local devices, which cannot robustly infer nuanced thought content or user intent from noisy biological signals such as brainwaves. As a result, these systems frequently misinterpret user intent, exhibit unstable behavior, or require extensive manual calibration, thereby reducing reliability and responsiveness.

Second, conventional architectures typically separate low-level signal analysis from higher-level natural language processing in an ad hoc manner. There is no tightly integrated, machine-learning-based pipeline that (i) continuously acquires time-series biological signals from a measurement apparatus via wireless communication, (ii) preprocesses and classifies the signals on a remote information processing apparatus using trained models, and (iii) automatically generates structured prompt sentences for a generative AI model to produce natural language output. This lack of integration leads to increased latency, redundant data transformations, and inefficient resource utilization across devices.

Third, many systems are not designed as computer-technical solutions to optimize end-to-end data flow and computation across a distributed environment including a measurement apparatus, a server, and a user terminal. For example, they do not adapt prompt generation based on model-estimated user states, and they do not coordinate actions such as application control on the user terminal in a way that is systematically derived from probabilistic inference over biological signals. As a consequence, communication overhead, processing bottlenecks, and inconsistent user experiences arise.

Fourth, current approaches do not adequately utilize generative AI models as programmable components in the signal-processing pipeline. Typically, natural language generation or translation is handled independently of low-level signal interpretation, and the content delivered to the user does not reflect real-time inference over their biological signals in a manner that is explicitly shaped by programmatically generated prompt sentences. This prevents efficient reuse of generative AI computation and makes it difficult to flexibly extend the system to multi-language interaction or context-sensitive responses.

Accordingly, there is a need for an improved computer-implemented system and server-side processing architecture that: (i) reliably acquires and preprocesses biological signals from a measurement apparatus, (ii) uses machine learning to infer user thought content and state, (iii) programmatically constructs prompt sentences tailored to the inferred thought content, (iv) invokes a generative AI model as a controlled computational component for natural language generation or translation, and (v) issues command information in coordination with natural language responses to drive application behavior on a user terminal. Such a system should improve the overall functioning of the computer system itself, including reduced latency, improved accuracy and stability of inference, and more efficient use of network and processing resources in a distributed environment.

The specific processing by the specific processing unit 290 of the data processing device 12 in Example 1 is realized by the following means.

The present invention provides a server comprising a processor configured to receive, via a network, digital signals representing biological signals acquired from a measurement apparatus through wireless communication, perform signal processing and machine learning processing on the digital signals to identify thought content of a user, determine, based on the identified thought content, information to be provided to the user or an operation to be executed, generate a prompt sentence that instructs a generative AI model to perform a conversion for expressing the information or the operation in natural language, control the generative AI model, executed as a program on at least one processing apparatus, to generate response information in natural language by using the prompt sentence as input, and transmit the response information and command information corresponding to the thought content to a user terminal so that the user terminal executes an application program based on the command information and presents at least one of a processing result of the application program or the response information to the user. This enables an integrated, computer-implemented pipeline that improves the technical performance of the system by efficiently transforming noisy biological signals into structured commands and natural language outputs, reducing end-to-end latency, enhancing accuracy and stability of user-intent inference, optimizing distribution of computation between server and terminal, and enabling flexible, multi-language interaction controlled through programmatically generated prompt sentences for a generative AI model.

The term “system” refers to an arrangement of one or more processing apparatuses, communication apparatuses, and terminal apparatuses that cooperate via at least one network to execute the functions described in the claims.

The term “processor” refers to a hardware processing unit, such as a central processing unit or a graphics processing unit, or a combination thereof, that executes instructions to perform signal processing, machine learning, prompt generation, natural language generation control, and communication control.

The term “biological signals” refers to time-varying physical signals generated by a living body, including but not limited to brainwave signals, that can be acquired by a measurement apparatus and converted into digital signals for processing.

The term “measurement apparatus” refers to a hardware device configured to detect biological signals from a user, convert the detected signals into digital signals, and transmit the digital signals via a communication interface.

The term “digital signals” refers to data representing sampled and quantized values of biological signals, formatted as discrete numerical values suitable for processing by a processor.

The term “wireless communication” refers to a mode of data transmission using electromagnetic waves without a wired physical connection, implemented by a communication protocol between the measurement apparatus and another apparatus.

The term “network” refers to a communication infrastructure, including at least one of a local area network, a wide area network, or a public communication network, that enables data exchange between the measurement apparatus, an information processing apparatus, and a user terminal.

The term “information processing apparatus” refers to a computing apparatus, such as a server, that includes at least one processor and memory, and is configured to perform signal processing, machine learning, prompt generation, and interaction with a generative AI model.

The term “signal processing” refers to computational operations applied to digital signals, including at least one of filtering, normalization, segmentation, and feature extraction, to prepare the signals for machine learning processing.

The term “machine learning processing” refers to computational operations performed using a trained model, including operations that map input digital signals to output values representing estimated thought content or user states based on learned parameters.

The term “thought content” refers to an inferred mental state or intention of a user, represented as one of multiple predefined categories, derived from biological signals by machine learning processing.

The term “information to be provided to the user” refers to content, such as recommendations, notifications, or explanations, that is selected or generated based on the thought content and is intended to be presented to the user.

The term “operation to be executed” refers to an action to be triggered in an apparatus, including at least opening, controlling, or configuring an application program on a user terminal, determined based on the thought content.

The term “prompt sentence” refers to a structured natural language or machine-readable instruction sequence that specifies to a generative AI model what type of natural language output, including content and format, is to be generated.

The term “generative AI model” refers to a machine learning model configured to generate natural language text or other content based on input tokens or prompt sentences, using learned parameters obtained from training on data.

The term “conversion for expressing the information or the operation in natural language” refers to a process in which the generative AI model generates natural language text that describes, explains, or instructs the information to be provided or the operation to be executed.

The term “response information” refers to natural language text or other output content generated by the generative AI model in response to a prompt sentence, to be presented to the user.

The term “command information” refers to control data that specifies an operation to be executed on a user terminal, such as identification of an application program and parameters for controlling execution of the application program.

The term “user terminal” refers to an end-user device, such as a mobile computing apparatus or a personal computing apparatus, configured to communicate with the information processing apparatus, execute application programs, and present information to the user.

The term “application program” refers to software executed on the user terminal that provides a specific function or service, and that can be launched or controlled based on command information received from the information processing apparatus.

The term “output apparatus” refers to a hardware component of the user terminal, such as a display or an audio output device, that presents visual or auditory information to the user.

The term “data pre-processing” refers to a sequence of computational steps performed before machine learning processing, including at least temporal segmentation, spatial organization, filtering, scaling, and transformation of digital signals into model input tensors.

The term “temporal features” refers to characteristics of biological signals that vary over time, such as patterns, trends, or periodic components within a time window, used as inputs to machine learning processing.

The term “spatial features” refers to characteristics of biological signals across multiple measurement channels or locations, representing relationships or distributions over measurement points, used as inputs to machine learning processing.

The term “probability distribution corresponding to a plurality of thought states” refers to a set of numerical values produced by a machine learning model, each value indicating a likelihood that the biological signals correspond to a respective thought state.

The term “behavior request of the user” refers to a type of user intention, inferred from biological signals, that implies a desired action or command, such as a request to start an application or to receive specific content.

The term “type of the determined thought content” refers to a categorical classification label assigned to the thought content, indicating a particular class of user intention or mental state.

The term “state of the user” refers to an inferred condition of the user, such as relaxed, focused, or neutral, estimated from biological signals and used to condition subsequent processing, including prompt generation.

The term “dynamically change content of the prompt sentence” refers to modifying wording, structure, or parameters of a prompt sentence at runtime, based on the determined thought content and user state.

The term “natural language text in a plurality of languages” refers to natural language strings expressed in two or more human languages, generated by or through the generative AI model.

The term “translation between different languages” refers to converting natural language content expressed in a first language into equivalent natural language content expressed in a second language, using a generative AI model.

The term “present the response information in the plurality of languages simultaneously or sequentially” refers to outputting natural language texts in two or more languages either at the same time or in a predefined order via the output apparatus of the user terminal.

In one embodiment, the system includes a server, at least one terminal, and at least one measurement apparatus configured to acquire biological signals from a user. The user wears a brainwave measurement apparatus that includes a plurality of electrodes, an analog front-end circuit, an analog-to-digital converter, and a wireless communication module. The measurement apparatus detects brainwave signals (EEG signals) from the scalp, converts the analog signals into digital samples, and transmits the digital samples via short-range wireless communication to the terminal.

The terminal operates as an intermediary communication and control device between the measurement apparatus and the server. The terminal includes a processor, a memory, a wireless communication interface for short-range communication, a network communication interface for wide-area communication, an output apparatus such as a display and a speaker, and an input apparatus such as a touchscreen. The terminal executes an application program that manages pairing with the measurement apparatus, acquisition of digital brainwave data, buffering of the data in a specified data structure, and forwarding of the buffered data to the server via a network.

The server operates as an information processing apparatus and includes at least one central processing unit, at least one graphics processing unit or other hardware accelerator, a main memory, a storage device, and a network interface. The server executes a plurality of software modules, such as an operating system, a network server program, a signal processing module, a machine learning inference module, a prompt generation module, a generative AI model control module, and an application control interface module. The server uses, for example, a general-purpose operating system such as a server-grade operating system, and a web application framework implemented in a programming language such as Python. The server stores the signal processing and machine learning inference modules as executable programs in the storage device, and loads them into the main memory for execution by the central processing unit and, when available, by the graphics processing unit.

The server receives, from the terminal, digital signals that represent brainwave activity of the user. The server stores the received samples in a structured format, for example as multi-dimensional arrays or tensors indexed by user identifier, channel index, and time index. The server applies signal processing operations such as band-pass filtering, notch filtering, baseline correction, and normalization to the digital signals. The server may use numerical computation libraries such as a numerical array processing library and a scientific signal processing library to implement these operations. The band-pass filtering emphasizes relevant frequency bands associated with cognitive states, and the notch filtering removes electrical noise such as power line interference.

The server segments the filtered signals into time windows of fixed duration, for example in the order of several hundred milliseconds to several seconds, and organizes the resulting data into a three-dimensional tensor of the form [window index, channel index, time sample index]. The server further computes temporal features, such as short-time Fourier transform coefficients or wavelet transform coefficients, and spatial features, such as channel-wise covariance matrices or spatially filtered components, and embeds these features into the tensor representation. This structured representation is specifically designed to match the expected input format of a trained neural network model and to improve the efficiency of memory access and parallel computation on the graphics processing unit.

The server uses a trained machine learning model implemented by a neural network framework such as a deep learning library to infer thought content of the user from the processed tensors. The server, for example, loads a model that combines convolutional layers and recurrent layers. The convolutional layers process the time dimension and channel dimension to extract local temporal and spatial patterns associated with specific brain states. The recurrent layers, such as gated recurrent units or long short-term memory units, capture longer temporal dependencies across consecutive windows. The server configures the neural network with specific layer sizes, activation functions, and regularization parameters to optimize performance for brainwave classification.

The server trains the model in advance using a dataset of labeled brainwave recordings. The server uses a supervised learning algorithm that minimizes a loss function such as cross-entropy loss between predicted thought state probabilities and ground-truth labels. The server updates model weights by a gradient-based optimization algorithm such as stochastic gradient descent with momentum or adaptive moment estimation. The server may augment the training data by adding small amounts of noise, time shifting, or scaling to improve robustness. During training, the server monitors validation accuracy and loss to prevent overfitting and to determine an appropriate stopping point.

The server uses the trained model at inference time to compute, for each incoming window tensor, a probability distribution over a set of predefined thought states. For example, the thought states may include “requesting music playback,” “requesting rest,” “requesting concentration mode,” “neutral,” and others. The server applies a softmax layer at the output of the neural network to obtain the probability distribution. The server then aggregates the probability distributions across consecutive windows and applies a temporal smoothing algorithm or a majority-vote rule to stabilize the final classification. This stabilizing step reduces rapid fluctuations caused by transient noise and thus improves the precision and reliability of the inferred thought content.

The server determines a thought content representing a behavior request of the user based on the probability distribution. When the probability for a particular thought state exceeds a predetermined threshold and remains stable for a predetermined time, the server decides that the user intends the corresponding behavior request. The server represents this decision as a structured record including at least a thought label, a confidence value, a time stamp, and a user identifier.

The server generates a prompt sentence for a generative AI model based on the determined thought content and, optionally, on additional context such as user history, user preferences, and current environmental conditions. The server constructs the prompt sentence as natural language text with a specific structure designed to guide the generative AI model toward a desired type of output. The server may adapt the wording, length, and included parameters of the prompt sentence dynamically. For example, when the server detects that the user is relaxed and has a “requesting music playback” thought content, the server generates a prompt sentence such as:
“The user's EEG pattern indicates a relaxed state and a desire to listen to music. Propose three playlist ideas (title and one-sentence description for each) that would help the user maintain a calm, relaxed mood.”

The server, in another example, generates a prompt sentence that emphasizes the technical state derived from the brainwave analysis, such as:
“Based on EEG data classified as a ‘focus’ state, describe in concise natural language how to adjust audio settings and music selection to support sustained concentration.”

The server provides the prompt sentence as input to a generative AI model that is executed either on the same server or on an external processing apparatus. The generative AI model may be, for example, a transformer-based language model configured to generate natural language outputs from prompt sentences. The server sends the prompt sentence as a sequence of tokens and configures inference parameters such as maximum output length, sampling temperature, and top-k or top-p sampling thresholds. The server then receives the generated natural language response from the generative AI model and stores it as response information.

The server uses the response information in parallel with command information derived from the thought content. For example, when the thought content corresponds to a music playback request, the server generates command information including a command type such as “open music application,” a target application identifier, and optional parameters such as playlist identifiers, search keywords, or audio profile settings. The server sends, via the network, both the response information and the command information to the terminal associated with the user.

The terminal receives the response information and the command information and parses them using a data structure such as a key-value mapping. The terminal determines, based on the command information, which application program to launch or control. The terminal uses an application programming interface of the operating system to start the corresponding application program, pass appropriate parameters, and, if necessary, adjust system-level settings such as volume, display mode, or notification settings. For example, the terminal launches a media playback application with a query that corresponds to the recommended playlist or music genre suggested by the server.

The terminal presents the response information to the user via the output apparatus. In one example, the terminal displays a text message on the screen, such as:
“We detected that you want to listen to music and relax. Suggested playlists: ‘Deep Calm Waves’, ‘Evening Soft Lights’, and ‘Slow Breaths’. Would you like to start playback now?”

In another example, the terminal uses a text-to-speech engine to convert the response information into voice output and plays the audio through a speaker, thereby allowing hands-free interaction. The terminal also presents interactive options such as buttons for “Play now,” “Show other options,” or “Cancel,” and, based on user input, sends feedback information to the server, which can be used for further model refinement.

The server improves computer technology by using the specific combination of hardware-assisted signal preprocessing, structured tensor representations, and neural network architectures to optimize the inference pipeline. By performing filtering, normalization, and feature extraction in a manner tailored to the subsequent neural network layers, the server reduces the dimensionality of the input, suppresses irrelevant noise, and concentrates the computational workload on discriminative patterns. This design reduces the number of operations required per inference and allows efficient batch processing on the graphics processing unit, thereby reducing processing time per window and enabling near real-time response.

The server further improves the accuracy and stability of inference by using probabilistic aggregation and temporal smoothing over consecutive windows. This approach reduces misclassification due to transient artifacts and yields a more consistent estimate of thought content. As a result, the overall system achieves higher precision in mapping noisy brainwave signals to discrete commands, which enables reliable automatic control of external applications and devices.

The server also improves communication efficiency by structuring the data exchange between the measurement apparatus, the terminal, and the server. The server causes the terminal to transmit compressed, windowed data rather than continuous raw streams, allowing the network to transport only the necessary segments. On the downstream path, the server transmits compact command information and human-readable response information, thereby reducing unnecessary data transfer. These configurations decrease network load and latency in a distributed environment.

The server generates prompt sentences in a programmatic manner that is directly conditioned on the inferred thought content and user state. This design differs from merely replacing human authorship with automated text generation. The prompt sentences encode internal state information from the brainwave-based classifier into structured natural language instructions for the generative AI model. This integration allows the generative AI model to function as a controllable component of the technical pipeline rather than as a generic text generator. As a result, the system can systematically adjust the style, content, and language of natural language outputs in response to real-time neural signal analysis, which is not achievable by manual prompt design at human speed or scale.

The server, in some embodiments, supports multi-language interaction. The server generates prompt sentences that instruct the generative AI model to output text in multiple languages or to translate content between languages. For example, when the server determines that the user is an advanced bilingual speaker and has a “requesting concentration mode” thought content, the server generates a prompt sentence such as:
“Generate two short focus-mode instructions, one in English and one in another specified language, that guide the user to maintain deep concentration while listening to instrumental music.”

The terminal then presents both language versions either simultaneously on a split display or sequentially using a swipe gesture. This configuration expands the utility of the system for multilingual users and assists in maintaining consistent interaction quality across languages. The server may be configured with alternative neural network architectures depending on hardware constraints and application requirements. In one variation, the server uses a purely convolutional neural network with temporal and spatial filters. In another variation, the server uses a hybrid architecture combining convolutional layers with attention mechanisms to focus on salient portions of the signal. The server may also vary hyperparameters such as number of layers, number of channels per layer, kernel sizes, and dropout rates. These alternative embodiments share the characteristic that they operate on structured representations of brainwave data and produce probability distributions over thought states, but differ in their specific architectural design and resource usage.

The terminal may also take alternative forms. In one embodiment, the terminal is a handheld smart device with a touchscreen. In another embodiment, the terminal is a head-mounted display or a vehicle-mounted terminal. In still another embodiment, the terminal is a stationary computing device connected to multiple output apparatuses. In each case, the terminal executes an application program that receives command information and response information from the server, controls local applications accordingly, and presents outputs to the user.

The system as a whole is not limited to music-related control. In another concrete example, the user focuses on a task and the server infers a “requesting concentration mode” thought content. The server then generates command information instructing the terminal to dim the display, silence non-critical notifications, and open a productivity application. The server further generates a prompt sentence such as:
“The user's EEG pattern indicates a high concentration state and a desire to maintain focus. Provide brief, encouraging guidance and suggest environmental settings (screen brightness, notification level, background sound) to support sustained focus.”

The generative AI model generates text describing recommended settings and motivational instructions, and the terminal partially automates the environment configuration according to the command information. In this way, the system actively controls device behavior based on brainwave-derived intent, thereby producing a tangible technical effect in the physical operation of the terminal.

By integrating detailed signal processing, specifically designed neural network inference, dynamic prompt sentence generation, and coordinated application control, the server and the terminal cooperate to achieve improvements in processing speed, accuracy, and communication efficiency that are not merely an automation of human mental steps. The server executes specialized algorithms and uses non-conventional data structures that are tailored to brainwave data and generative AI interaction, and thus improves the functioning of the computer system itself in a distributed, real-time setting.

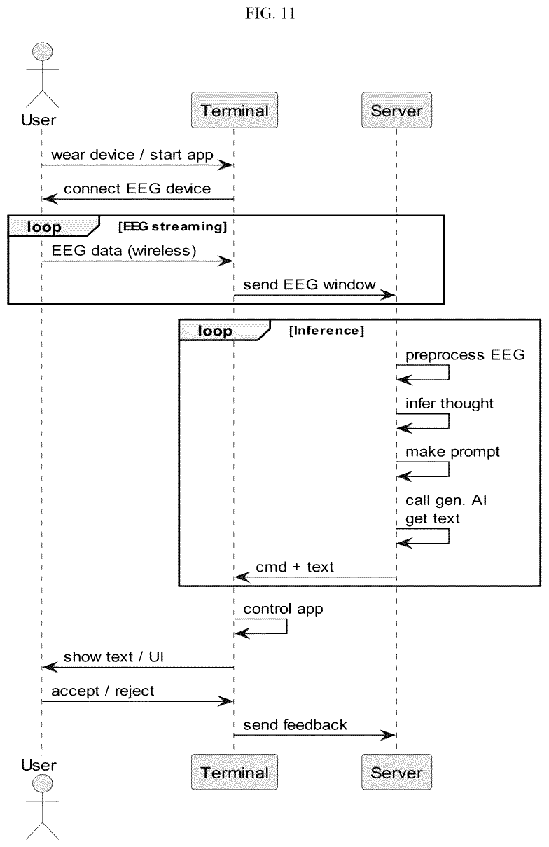

The following describes the processing flow using FIG. 11.

### Step 1

The user wears a measurement apparatus.

The user places an EEG headset on the head, adjusts electrodes so that they contact the scalp, and turns on the power of the headset.

Input: the user's brain electrical activity.

Output: analog EEG signals at each electrode.

The measurement apparatus converts the analog EEG signals into digital samples by using an internal analog-to-digital converter and stores the samples in an internal buffer for wireless transmission.

### Step 2

The terminal establishes a wireless connection to the measurement apparatus.

The terminal activates a short-range wireless interface, discovers the measurement apparatus, and performs a pairing procedure.

Input: device identification data broadcast by the measurement apparatus.

Output: a wireless communication session between the terminal and the measurement apparatus.

The terminal invokes an operating system API to subscribe to a data characteristic exposed by the measurement apparatus so that EEG data packets can be received asynchronously.

### Step 3

The terminal receives and buffers digital EEG data.

The terminal continuously receives EEG data packets from the measurement apparatus through the wireless interface and decodes packet headers and payloads.

Input: EEG data packets containing channel indices, sample values, and timestamps.

Output: a structured buffer of EEG samples organized by channel and time.

The terminal writes each sample into a circular buffer in memory, indexed by a time index and channel index, and discards or overwrites the oldest data when the buffer is full.

### Step 4

The terminal groups EEG data into windows and prepares it for transmission.

The terminal monitors the buffer and extracts fixed-length segments of EEG data corresponding to predefined time windows, for example one second of data.

Input: continuous EEG samples stored in the circular buffer.

Output: a data structure representing a windowed EEG segment, including metadata such as user identifier, sampling rate, and channel labels.

The terminal serializes the windowed EEG segment into a compact format and optionally compresses the serialized data to reduce its size.

### Step 5

The terminal sends EEG windows to the server over a network.

The terminal uses a network communication interface to establish a secure session with the server and transmits each prepared EEG window as soon as it is available.

Input: serialized and optionally compressed EEG windows.

Output: network packets sent to the server that contain user identifiers, timestamps, and EEG window data.

The terminal manages retransmission or queuing when the network is unstable and confirms reception with the server via acknowledgment messages.

### Step 6

The server receives and stores the EEG windows.

The server listens on a network interface, accepts connections from terminals, and reconstructs received packets into complete EEG window messages.

Input: network packets carrying serialized EEG windows.

Output: EEG window objects stored in server memory with associated user identifiers and timestamps.

The server validates message integrity and schema, then writes each EEG window into a temporary storage queue and, optionally, into a persistent database.

### Step 7

The server performs signal preprocessing on the EEG windows.

The server retrieves EEG windows from the temporary storage queue and applies digital signal processing operations to each channel.

Input: raw EEG windows organized by channel and time.

Output: filtered and normalized EEG windows ready for feature extraction.

The server applies a band-pass filter to keep frequencies in a target range, applies a notch filter to remove power-line noise, subtracts a baseline value from each channel, and normalizes each channel so that its values have a standardized range.

### Step 8

The server extracts temporal and spatial features and forms tensors.

The server converts the preprocessed windows into feature representations suitable for machine learning.

Input: filtered and normalized EEG windows.

Output: multi-dimensional tensors containing temporal features and spatial features for each window.

The server calculates features such as power spectral density, transforms the signals into a frequency domain or time-frequency domain, computes relationships between channels, and arranges the resulting values into a tensor with dimensions corresponding to batch index, channel index, and time or feature index.

### Step 9

The server infers thought content using a trained neural network.

The server loads a trained neural network model into memory and performs forward propagation on the feature tensors.

Input: tensors that represent EEG features for a series of windows.

Output: probability distributions over predefined thought states for each window.

The server multiplies the tensors by learned weight matrices, applies convolution or recurrent operations across time and channels, applies non-linear activation functions, and applies a final softmax layer to obtain probabilities for each thought state.

### Step 10

The server aggregates probabilities and determines a final thought content.

The server combines the probability distributions across a sequence of windows to stabilize the inference result.

Input: a sequence of probability vectors for consecutive windows.

Output: a single thought content label and a confidence value for the user at a given time.

The server calculates statistics such as moving averages of probabilities, applies thresholds, and selects the thought state that remains dominant over a defined time interval as the final thought content.

### Step 11

The server determines an operation or information to provide based on the thought content.

The server consults internal rules or configuration mappings to convert the thought content into a behavior request and corresponding operation.

Input: the thought content label, confidence value, and user context information.

Output: an operation specification that describes which application should be controlled and what parameters should be used.

The server, for example, maps a “music request” thought content to an operation specification that includes an instruction to start a media application and recommended content type, such as relaxing music.

### Step 12

The server generates a prompt sentence for a generative AI model.

The server constructs a natural language instruction that reflects the thought content and user state and that instructs the generative AI model to generate a specific kind of text.

Input: the operation specification, the inferred user state, and user preference data.

Output: a prompt sentence in natural language formatted to guide the generative AI model.

The server, for example, creates a prompt sentence such as: “The user's EEG pattern indicates a relaxed state and a desire to listen to music. Propose three playlist ideas (title and one-sentence description for each) that would help the user maintain a calm, relaxed mood.”

### Step 13

The server invokes the generative AI model to generate response information.

The server sends the prompt sentence to the generative AI model and configures inference parameters such as maximum output length and sampling conditions.

Input: the prompt sentence and generation parameters.

Output: generated natural language text that responds to the prompt sentence.

The server receives the generated text, analyzes its structure if necessary, and stores it as response information associated with the user and the current thought content.

### Step 14

The server composes command information and packages the results.

The server builds a data structure that combines the operation specification and the generated response information for delivery to the terminal.

Input: the operation specification and the response information.

Output: a combined message including command information and human-readable natural language content.

The server encodes the message in a transport format suitable for the network, adds identifiers and timestamps, and queues the message for transmission to the terminal.

### Step 15 T

he server sends the combined message to the terminal.

The server transmits the combined message through the network interface to the terminal associated with the user.

Input: the combined message containing command information and response information.

Output: network packets addressed to the terminal carrying the combined message.

The server manages acknowledgment handling and retransmission to ensure that the terminal receives the message correctly.

### Step 16

The terminal receives and interprets the combined message.

The terminal reads the message from the network, decodes it, and separates the command information from the response information.

Input: a combined message from the server.

Output: internal representations of the command information and the response information.

The terminal inspects the operation type, target application identifier, and any parameters in the command information and prepares to perform the requested operations.

### Step 17

The terminal controls an application program based on the command information.

The terminal uses an operating system interface to launch or control a specified application program in accordance with the command information.

Input: operation type, application identifier, and application parameters from the command information.

Output: an executed or updated application state, such as a running media player configured with recommended content.

The terminal may, for example, start a media playback application, send it a request to open a recommended playlist, and adjust system settings such as volume to match the inferred user state.

### Step 18

The terminal presents the response information to the user.

The terminal displays the generated natural language text on a screen and, in some cases, converts it into speech for audio output.

Input: the response information text.

Output: visual and/or audio output that communicates recommendations or explanations to the user.

The terminal shows, for instance, a message containing suggested playlists and provides interface elements that allow the user to accept or reject the recommendation.

### Step 19

The user reacts to the presented information and controls the system.

The user reads or listens to the response information and interacts with the terminal via input mechanisms such as tapping buttons or voice commands.

Input: application state and user interface elements displayed on the terminal.

Output: user input actions that confirm, modify, or cancel the recommended operation.

The user, for example, taps a “Play now” button to start music playback or taps a “Cancel” button if the recommendation is not desired.

### Step 20

The terminal sends user feedback to the server.

The terminal packages the user's input, including the association with the corresponding thought content and time, into a feedback message.

Input: user input actions and identifiers of the current session and thought content.

Output: a feedback message that indicates acceptance or rejection of the operation and other user responses.

The terminal transmits this feedback message to the server, where it can be stored and later used to improve the machine learning model or adjust rule parameters.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional information provision systems, including recommendation engines and content delivery platforms, are typically designed to operate on explicit user inputs such as search queries, menu selections, or simple preference settings. These systems primarily rely on static profiles, coarse interaction logs, and heuristic-ranking techniques, and thus are not capable of capturing rapid, fine-grained changes in a user's cognitive state or emotional state. As a result, such systems often deliver content that is misaligned with the user's real-time needs, for example, providing stimulating content when the user is mentally fatigued, or focusing content when the user intends to relax. This mismatch leads to reduced usability, decreased user satisfaction, and inefficient consumption of computational and network resources due to unnecessary content retrieval and presentation cycles.

In parallel, recent advances in biological signal measurement, such as brainwave acquisition by electroencephalographic devices, enable real-time acquisition of high-dimensional time-series data that reflects a user's internal cognitive and emotional states. However, typical computer systems are not architected to efficiently integrate such biological signals into the software stack of content recommendation and generation. In particular, existing architectures do not define concrete mechanisms for transforming raw time-series biological signals into structured “thought information,” for coupling this thought information with generative artificial intelligence models via prompt sentences, and for using the outputs of such models to drive low-latency, personalized content selection and presentation. Without such mechanisms, the hardware and software resources that perform signal processing, inference, and content generation cannot be coordinated effectively, and the potential of biological-signal-driven personalization remains unrealized.

Furthermore, conventional systems that utilize machine learning and generative models often treat those models as isolated components, invoked with static or manually crafted prompts. Such systems do not provide a feedback loop in which user evaluations of generated content and context information (for example, time information and usage-context information) are systematically associated with the underlying thought information and content choices. Consequently, the system cannot adapt its internal models, prompt-generation logic, or control parameters of the generative AI model in a principled, data-driven manner. This results in suboptimal learning efficiency, stagnation of recommendation quality, and unnecessary consumption of computational cycles for executing ineffective model inferences.

Additionally, known systems typically lack an integrated mechanism that coordinates (i) preprocessing pipelines for biological signals, (ii) statistical learning models that infer cognitive or emotional states, (iii) prompt-data generation mechanisms that control generative AI models, and (iv) downstream content retrieval and ranking processes. The absence of such an end-to-end architecture leads to fragmented implementations, redundant data transformations, and excessive inter-process communication overhead. From a computer-technology perspective, there is a need for a unified system architecture in which a processor orchestrates these functional blocks so that memory usage, processing latency, and network traffic are reduced while personalization accuracy is increased.

Accordingly, there is a demand for an improved computer-implemented system that, by using a processor, (i) converts raw biological time-series data into machine-learning input data, (ii) determines thought information representing a user's cognitive or emotional state, (iii) generates structured prompt data for a generative AI model, (iv) obtains and interprets content specification information or response information from the generative AI model, and (v) determines, delivers, and adaptively refines personalized presentation content. Such a system should further implement closed-loop learning in which user feedback is accumulated as learning data for updating both the statistical learning model and the prompt-generation process, thereby improving, over time, the technical performance of the recommendation pipeline, including prediction accuracy, resource utilization efficiency, and response time.

The specific processing by the specific processing unit 290 of the data processing device 12 in Application Example 1 is realized by the following means.

The present invention provides a server comprising a processor and a memory storing instructions that, when executed by the processor, cause the server to measure a biological signal of a user and acquire the biological signal as time-series data; to preprocess the time-series data and convert the time-series data into machine-learning input data by performing at least frequency-component extraction and feature generation on the time-series data; to classify, by using a statistical learning model, the machine-learning input data to determine thought information by identifying at least one of a thought content and an emotional state of the user; to determine, by referring to the thought information and profile information of the user, an information type and an information output format corresponding to the thought information, and generate prompt data including a prompt sentence for instructing a conversion of the thought information into natural language; to input the prompt data into a generative artificial intelligence model and instruct the generative artificial intelligence model to generate at least one of response information in natural language and content specification information; to acquire, on the basis of at least one of the content specification information and the response information, candidate content from at least one of an external information providing apparatus and an information storage apparatus, and determine personalized presentation content by selecting or composing the candidate content based on at least the thought information and history information of the user; to convert the personalized presentation content into output data, transmit the output data to a terminal device, and cause the terminal device to present the personalized presentation content as at least one of visual information and auditory information to the user; and to acquire, from the terminal device, feedback information including at least one of operation information and evaluation information of the user with respect to the personalized presentation content, store the feedback information in association with the thought information and the personalized presentation content, and accumulate the feedback information as learning data for updating at least one of the statistical learning model and a generation process of the prompt data. This enables a computer-centric improvement in which the server implements an integrated, closed-loop processing pipeline that transforms raw biological time-series data into optimized prompt sentences and personalized content, thereby enhancing state-detection accuracy, reducing ineffective model inferences, improving utilization of processing and network resources, and adaptively increasing the relevance and timeliness of content delivered to the user.

The term “biological signal” refers to an analog or digital signal representing a physiological activity of a living subject, including but not limited to brainwave activity, cardiac activity, muscle activity, or skin conductance, which can be measured by a sensor and processed by an electronic apparatus.

The term “time-series data” refers to a sequence of numerical values or vectors that are ordered in time and sampled at one or more sampling intervals, representing the temporal evolution of a biological signal or a derived quantity.

The term “preprocess” refers to performing one or more signal-processing or data-processing operations, such as filtering, noise removal, normalization, segmentation, and feature extraction, on raw data to convert the raw data into a form suitable for input to a machine-learning model or another computational module.

The term “frequency-component extraction” refers to a process of analyzing a time-domain signal to obtain information about its spectral content, including but not limited to amplitude or power within one or more frequency bands, using methods such as Fourier transforms or spectral density estimation.

The term “feature generation” refers to computing derived values, vectors, or structures from raw or preprocessed data, where the derived values capture characteristics of the data, such as statistical moments, spectral powers, or temporal patterns, for use as input variables to a machine-learning model.

The term “machine-learning input data” refers to data that has been preprocessed and structured, including generated features, in a numerical or symbolic representation that conforms to the input format required by a machine-learning model.

The term “statistical learning model” refers to a computational model that estimates parameters, probabilities, or mappings from input data to output data based on statistical patterns learned from example data, including but not limited to neural networks, support vector machines, decision trees, and probabilistic graphical models.

The term “thought information” refers to structured information representing an internal state of a user, including at least one of a cognitive intention, a mental activity, and an emotional state, determined from biological signals or related data.

The term “thought content” refers to a component of thought information that expresses a user's cognitive intention or focus, such as a desire to relax, concentrate, or engage in a particular type of activity.

The term “emotional state” refers to a component of thought information that expresses a user's affective condition, such as calmness, stress, excitement, or boredom, inferred from biological signals or related data.

The term “profile information of the user” refers to information associated with a user that is stored in a storage apparatus, including but not limited to demographic data, historical usage data, preference settings, and interaction logs.

The term “information type” refers to a classification of content or data to be provided to a user, such as audio content, visual content, text content, or mixed media content.

The term “information output format” refers to a specification of a representation or structure in which content is provided, such as a playlist, a list of items, a narrative text, a summary, or structured metadata.

The term “prompt data” refers to data that includes a prompt sentence or a set of instructions formulated in natural language or a structured format, designed to control or guide a generative AI model to produce a desired output.

The term “prompt sentence” refers to a sequence of words in natural language that describes an instruction, constraint, or context for a generative AI model, including conditions related to content type, mood, length, output format, or metadata.

The term “generative artificial intelligence model” refers to a computational model configured to generate data, including natural-language text or structured information, in response to an input such as a prompt sentence, based on patterns learned from training data.

The term “response information” refers to natural-language text or equivalent output generated by a generative artificial intelligence model in response to input prompt data, and may include explanations, descriptions, or recommendations.

The term “content specification information” refers to information, often expressed in natural language or structured form, that describes characteristics of content to be generated or selected, including but not limited to genre, mood, duration, structure, and metadata fields.

The term “external information providing apparatus” refers to a network-accessible system or service that supplies content or data, such as a content server, a media distribution service, or an information retrieval system.

The term “information storage apparatus” refers to a storage subsystem or service, such as a database, a file storage system, or a data repository, configured to store and provide access to content or related data.

The term “candidate content” refers to one or more items of content, such as media items or data records, retrieved from an external information providing apparatus or an information storage apparatus, which are evaluated for potential presentation to a user.

The term “personalized presentation content” refers to content that has been selected, filtered, or composed based on at least thought information and user-related information, such that the content is tailored to the estimated state, preferences, or context of the user.

The term “history information of the user” refers to data that records past interactions of the user with content and the system, including but not limited to viewed items, played items, ratings, and feedback events.

The term “output data” refers to data formatted for transmission from the server to a terminal device, specifying content to be presented and optionally including control parameters or metadata for rendering.

The term “terminal device” refers to an electronic device operated by or near a user, such as a portable communication device, a wearable device, a computer, or a display apparatus, configured to present content and receive user input.

The term “visual information” refers to content presented in a form perceivable by sight, including images, video, text, graphics, or graphical user interfaces.

The term “auditory information” refers to content presented in a form perceivable by hearing, including speech, music, sound effects, or other audio signals.

The term “operation information” refers to data representing user actions performed via an input interface of a terminal device, such as button presses, cursor movements, touch inputs, or selection operations.

The term “evaluation information” refers to data that explicitly or implicitly represents a user's assessment of content, such as ratings, likes, dislikes, preference selections, or qualitative feedback.

The term “feedback information” refers to information obtained from a terminal device that includes at least one of operation information and evaluation information associated with presented content.

The term “learning data” refers to data stored for use in training or updating a machine-learning model or a related computational process, including associations among input features, intermediate representations, outputs, and feedback.

The term “generation process of the prompt data” refers to a sequence of operations executed by the processor to construct prompt data, including selection of parameters, inclusion of context information, and formulation of prompt sentences.

The term “output conditions” refers to constraints or requirements specified for a generative artificial intelligence model, such as desired output length, style, level of detail, or required fields.

The term “prohibition conditions” refers to constraints indicating elements or characteristics that a generative artificial intelligence model should avoid in its output, such as disallowed topics, genres, or stylistic features.

The term “time information” refers to data indicating temporal context, including at least one of absolute time, date, time of day, or elapsed time related to system operation or content usage.

The term “usage-context information” refers to information relating to the circumstances under which the system is used, including but not limited to device type, location category, user activity type, session duration, or concurrent applications.

The term “server” refers to a computing apparatus or a group of computing apparatuses that provide processing, storage, and communication functions, and that execute instructions to implement at least part of the system described herein.

The term “processor” refers to one or more processing units, such as a central processing unit, a graphics processing unit, or another programmable circuit, configured to execute instructions stored in a memory to perform operations on data.

The term “memory” refers to one or more computer-readable storage media, such as volatile memory, nonvolatile memory, or mass storage, configured to store data and instructions for access by a processor.

In one embodiment, a server, a terminal, and a biological-signal measurement device cooperate to implement the claimed system. The server includes at least one processor, a volatile memory, a nonvolatile storage device, and a network interface. The terminal includes at least one processor, a memory, a display device, an audio output device, and a user input interface. The biological-signal measurement device includes at least one sensor, an analog-to-digital converter, and a short-range communication interface.

The user wears the biological-signal measurement device on the head so that the device is positioned to measure brainwave activity. The device measures an analog biological signal corresponding to brainwave activity and digitizes the analog signal by using the analog-to-digital converter at a predetermined sampling frequency. The device transmits the digitized signal to the terminal over a wireless communication link, such as a Bluetooth or Wi-Fi link. The terminal executes an application program that runs on an operating system such as a mobile operating system or a desktop operating system. The terminal receives the digitized biological signal as time-series data and buffers the time-series data in a local memory. The terminal transmits the buffered time-series data to the server via a network using a secure transport protocol such as HTTPS. In this way, the server acquires the biological signal as time-series data in digital form.

The server executes software implemented, for example, in a programming language such as Python or another high-level language. The server stores and executes libraries for numerical computation and signal processing, such as a numerical array library and a scientific computation library. The server loads the received time-series data into an in-memory numerical array and applies preprocessing operations. The server applies at least one band-pass filter to remove very low-frequency components and very high-frequency components that are not relevant to cognitive state estimation. The server applies artifact-suppression procedures, such as separating independent components and attenuating components corresponding to eye movements and muscle artifacts, by using an independent component analysis algorithm implemented in a signal-processing library. The server normalizes each channel of the time-series data by subtracting a mean value and dividing by a standard deviation to form standardized samples.

The server converts the preprocessed time-series data into machine-learning input data. The server segments the time-series data into fixed-length windows and, for each window, computes frequency-domain features by applying a discrete Fourier transform or a fast Fourier transform. The server calculates power values in multiple frequency bands, such as delta, theta, alpha, beta, and gamma bands, by summing squared magnitudes of Fourier coefficients in corresponding frequency ranges. The server also computes statistical features such as mean, variance, skewness, and kurtosis of the time-domain signal within each window. The server concatenates these features into a feature vector for each time window. The server combines multiple feature vectors from multiple channels and multiple time windows into a feature tensor that conforms to an input shape expected by a statistical learning model.

In one embodiment, the server implements the statistical learning model as a deep neural network constructed by a machine-learning framework such as a neural-network library that supports tensor operations. The server defines the model as a sequence of layers, which may include one-dimensional convolutional layers that capture local temporal patterns in the feature tensor, recurrent layers such as gated recurrent units or long short-term memory units that capture temporal dependencies across windows, and fully connected layers that map the high-level representation to a probability distribution over discrete mental states. The server further configures one or more dropout layers and normalization layers to improve generalization and training stability. The server stores model parameters in a file, for example in a format used by the machine-learning framework, and loads the parameters into memory when starting an inference service.

The server determines thought information by inputting the machine-learning input data into the statistical learning model and executing a forward computation. The server obtains output values representing a probability for each mental-state class, such as a state indicating a desire to relax, a state indicating a desire to concentrate, a state indicating stress, and a state indicating boredom. The server selects the mental-state class that has a maximum probability and also obtains a confidence value based on the probability distribution. The server constructs thought information as structured data including at least one of a thought content representing a cognitive intention and an emotional state representing an affective condition, as well as the confidence value and optional latent features from an intermediate layer of the neural network.

The server stores profile information and history information of the user in a database. The server retrieves the profile information and the history information in response to determination of the thought information. The profile information includes preference categories, preferred media types, and user-specific constraints such as a maximum duration of content presentations. The history information includes records of previously presented content, feedback information associated with the content, and associated thought information previously inferred. The server determines an information type and an information output format by using a set of rules or a lightweight decision model that maps combinations of thought information and profile information to categories such as audio playlist, text summary, or mixed media collection. For example, when the thought information indicates a desire to relax, the server selects an audio playlist as the information type and a sequence of audio tracks with a total length of a predefined range as the information output format.

The server generates prompt data for a generative AI model by constructing a prompt sentence in natural language. The server uses a template-based prompt construction module that fills specific slots with current thought information, profile information, and desired content parameters. For example, when the thought information indicates a desire to relax, the server generates a prompt sentence such as:
“The user's brainwave-derived mental state indicates that the user wants to relax. Based on this state, recommend a 30-minute playlist of very relaxing ambient and soft piano music suitable for reducing stress and mental fatigue. Describe the overall mood and suggest several suitable sub-genres.”

When the thought information indicates a desire to concentrate, the server generates a prompt sentence such as:
“The user's brainwave-derived mental state indicates that the user wants to concentrate on a 60-minute work or study session. Based on this state, recommend instrumental music without lyrics that enhances focus and minimizes distraction. Specify suitable genres and describe the desired tempo and energy level.”

In another example, the server generates a prompt sentence that explicitly controls output format:
“The user's EEG patterns indicate the state ‘relax’ with high confidence. Using this state, recommend a 30-minute playlist of very relaxing ambient and soft piano music. Return a description of the playlist including a title, a detailed explanation of the mood, and a list of recommended genres.”

The server packages the prompt sentence and additional control parameters, such as maximum response length and desired level of detail, into prompt data and transmits the prompt data to a generative AI model. In one embodiment, the generative AI model is implemented as a transformer-based neural network that was trained on a large corpus of text. The server accesses the generative AI model via an application programming interface over a network. The server specifies decoding parameters, such as temperature, top-k value, and top-p value, to control the diversity and determinism of generated text.

The server receives response information from the generative AI model as natural-language text. The response information contains content specification information that describes, for example, target genres, desired tempo, recommended instruments, and narrative descriptions of mood. The server parses the response information using natural-language processing techniques such as pattern matching and keyword extraction, or by instructing the model, via the prompt sentence, to output content specification information in a semi-structured textual format that can be split into labeled sections. The server converts the extracted information into an internal structured representation containing fields such as content type, mood tags, target duration, tempo range, and preferred genres.

The server determines candidate content by querying at least one external information providing apparatus or an information storage apparatus. In one embodiment, the server communicates with a media service via a web application programming interface. The server constructs a query including fields such as desired genres, mood tags, and duration constraints derived from the content specification information. The server transmits the query and receives a list of candidate content items, each item including metadata such as title, length, genre, and unique identifier.

The server ranks the candidate content items according to a scoring function that depends on compatibility with the thought information, compatibility with the content specification information, and consistency with the history information. The server may implement the scoring function as a weighted sum of similarity measures, where weights may be learned based on past feedback information. The server selects one or more candidate content items having the highest scores or composes a new sequence of items, such as a playlist, that satisfies constraints such as target total length and diversity requirements. The selected or composed set of items constitutes personalized presentation content.

The server converts the personalized presentation content into output data for the terminal. The output data contains at least identifiers for content items, associated metadata such as titles and descriptions, and control information indicating in which order and with which playback parameters the terminal should present the content. The server transmits the output data to the terminal via the network.

The terminal receives the output data and stores it in a local memory. The terminal selects a presentation module based on the information type and information output format indicated in the output data. For audio content, the terminal uses an audio playback component provided by the operating system to stream the content from a media server or to play locally cached content, and the terminal displays an interface showing the title, description, and other metadata of the personalized presentation content on the display device. For visual content, the terminal uses a media rendering component to display images or video according to instructions in the output data.

The user perceives the personalized presentation content through auditory and/or visual channels. The user may interact with the content by using the user input interface of the terminal, for example by pressing a like or dislike button, adjusting a rating control, or selecting options indicating whether the content was helpful for relaxing or concentrating. The terminal records these interactions as operation information or evaluation information and associates them with identifiers of the personalized presentation content and the current thought information. The terminal transmits the resulting feedback information to the server.

The server receives the feedback information and stores it in association with the thought information and the personalized presentation content in a database. The server accumulates such associated records as learning data for improving the statistical learning model and for adjusting the generation process of the prompt data. The server periodically executes an offline training process or an online update process. In the training process, the server uses the learning data to compute gradients of a loss function, such as a cross-entropy loss for mental-state classification combined with a ranking loss for content selection. The server updates weights of the statistical learning model through gradient descent or a variant such as Adam optimization. The server may also update parameters of the scoring function used for ranking candidate content.

The server also refines the prompt-generation logic by analyzing correlations between specific prompt sentence patterns and resulting user feedback. For example, if content generated after including a condition “low energy and slow tempo” consistently receives higher ratings for concentration sessions, the server increases the likelihood of inserting such wording when the thought information indicates a need for high concentration with low stress. The server maintains a set of prompt templates and associated rule parameters, and updates these parameters based on aggregated statistics. Thus, the prompt-generation module evolves over time, improving the alignment between the generative AI model's outputs and user needs.

This integrated design provides several technical effects beyond mere automation of human decision-making. By performing domain-specific preprocessing, including frequency-component extraction and feature generation, the server transforms high-volume time-series data into compact feature tensors that reduce memory usage and computation cost for the statistical learning model. By using a neural-network architecture that combines convolutional and recurrent layers tuned for brainwave-derived signals, the server increases classification accuracy for thought information compared to generic classifiers, which in turn reduces misaligned content recommendations and avoids unnecessary invocations of the generative AI model.

By coupling the thought information to the generative AI model through structured prompt sentences, the server reduces the search space for the generative AI model and improves the relevance of generated content specification information. As a result, the server can reduce the number of interactions with external content services and network round trips that would otherwise be required to test multiple content candidates. The feedback loop, in which user evaluation is systematically tied to both the statistical learning model and the prompt-generation process, allows the server to optimize system-level performance metrics, such as response latency and recommendation precision, rather than simply reproducing a static recommendation rule.

The described architecture also improves computer-technology aspects such as data management and pipeline efficiency. The server enforces a particular data structure for intermediate representations, including standard shapes for tensors and schemas for thought information and content specification information, which reduces the overhead of serialization and parsing between modules. The modular decomposition into preprocessing, inference, prompt generation, generative-model interaction, content retrieval, ranking, and feedback assimilation allows the server to allocate computing resources dynamically, for example by assigning heavier GPU resources to inference and lighter CPU resources to parsing and prompt generation, thereby optimizing throughput and power consumption. In alternative embodiments, the server may utilize different types of biological signals, such as heart-rate variability or skin conductance, in addition to or instead of brainwave-derived signals. The server may adjust the feature generation stage to compute features specific to these signals, such as inter-beat intervals or conductance changes, and may modify the architecture of the statistical learning model accordingly. In another variation, the server may implement the generative AI model locally instead of accessing an external model via a network, and may store the model weights in the server's memory for low-latency inference. In still another embodiment, the terminal may perform part of the preprocessing, such as filtering and segmentation, to reduce network bandwidth requirements, and the server may perform only higher-level feature generation and inference. Conversely, in resource-rich environments, the server may centralize all heavy computations, and the terminal may function primarily as a data collection and presentation device. In each case, the same logical flow of transforming biological time-series data into thought information, generating prompt sentences, obtaining content specification information from a generative AI model, selecting personalized presentation content, and refining the models based on feedback remains applicable.

By the above configurations, the server, the terminal, and the user cooperate in a way that ties abstract data processing directly to control and optimization of real technical components, such as sensors, data transmission paths, processors, memories, and media rendering subsystems. The system thereby achieves improved accuracy and efficiency in translating biological signals into personalized content, improved utilization of computational and network resources, and reduced latency in responding to rapid changes in user cognitive and emotional states.

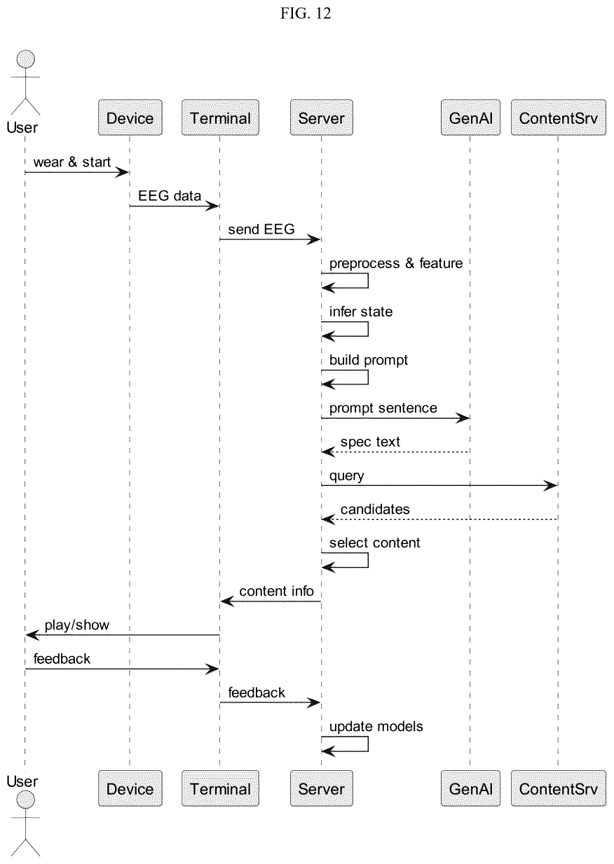

The following describes the processing flow using FIG. 12.

### Step 1

The user wears the biological-signal measurement device and activates the device. The input of Step 1 is the physical state of the user, including brain activity. The output of Step 1 is an analog biological signal representing the user's brainwave activity. The device converts the analog biological signal into a digital time-series signal by using an internal analog-to-digital converter and transmits the digital samples to the terminal through a short-range wireless link.

### Step 2

The terminal receives the digital samples from the biological-signal measurement device and buffers the samples in a local memory as time-series data.

The input of Step 2 is a stream of digitized biological-signal samples. The output of Step 2 is a structured sequence of samples, each associated with a timestamp and channel identifier. The terminal performs data framing by grouping samples into fixed-duration frames, attaches metadata such as device ID and sampling rate, and prepares the framed data for network transmission.

### Step 3

The terminal transmits the framed time-series data to the server via a network using a secure protocol.

The input of Step 3 is the framed time-series data stored in the terminal's memory. The output of Step 3 is a network message, for example an HTTPS request, containing the time-series data and session identifiers received by the server. The terminal serializes the data into a transport format, adds authentication tokens, and sends the message to a predefined server endpoint.

### Step 4

The server receives the network message from the terminal and stores the time-series data into a processing buffer.

The input of Step 4 is the network message including serialized time-series data and metadata. The output of Step 4 is an in-memory representation of the time-series data, such as a numerical array indexed by time and channel. The server validates the structure of the received data, verifies timestamps, checks channel counts, and writes the validated data into a queue or buffer for subsequent preprocessing.

### Step 5

The server performs preprocessing on the time-series data to generate cleaned signal data.

The input of Step 5 is the raw time-series data as stored in the server's buffer. The output of Step 5 is cleaned and normalized signal data for each channel. The server applies band-pass filters to remove undesired low-frequency drift and high-frequency noise, executes artifact-removal algorithms to suppress eye-blink and muscle artifacts, and performs normalization by subtracting a mean value and dividing by a standard deviation for each channel, thereby generating standardized signals suitable for feature extraction.

### Step 6

The server computes features from the cleaned signal data and constructs machine-learning input data.

The input of Step 6 is the cleaned, normalized time-series data. The output of Step 6 is a feature tensor that aggregates features across time windows and channels. The server segments the cleaned signals into overlapping or non-overlapping windows, applies spectral analysis such as fast Fourier transforms to each window, computes power values for multiple frequency bands, calculates time-domain statistics such as mean and variance, and concatenates these feature values into vectors. The server then combines the feature vectors across all channels and windows into a tensor that conforms to the input specification of a statistical learning model.

### Step 7

The server performs inference using a statistical learning model to determine thought information.

The input of Step 7 is the feature tensor representing the processed biological signals. The output of Step 7 is thought information, including at least one of a thought content and an emotional state, together with a confidence value. The server loads a trained neural-network model, such as a model composed of convolutional layers and recurrent layers, feeds the feature tensor into the model, executes a forward computation to obtain class probabilities, selects the mental-state class with the highest probability, and forms structured thought information that includes the selected state, the probability as confidence, and optional latent features from intermediate layers.

### Step 8

The server retrieves profile information and history information of the user and determines an information type and an information output format.

The input of Step 8 is the thought information and an identifier of the user. The output of Step 8 is a decision specifying an information type, such as audio content or visual content, and an information output format, such as a playlist or a text summary. The server queries a database for stored profile and history data, applies predefined mapping rules or a decision model that relates combinations of thought information and user characteristics to content categories, and selects appropriate content type and structural format parameters to be used in subsequent content generation.

### Step 9

The server generates prompt data including a prompt sentence for a generative AI model.

The input of Step 9 is the thought information, the determined information type and format, and user-related information such as preferences. The output of Step 9 is prompt data that contains at least one natural-language prompt sentence and associated control parameters. The server selects a template corresponding to the information type, fills slots in the template with the current mental state, desired duration, mood description, and constraints on content, and generates a prompt sentence, for example: “The user's brainwave-derived mental state indicates that the user wants to relax. Based on this state, recommend a 30-minute playlist of very relaxing ambient and soft piano music suitable for reducing stress and mental fatigue.” The server then bundles this sentence with parameters such as maximum response length and required details into prompt data.

### Step 10

The server transmits the prompt data to the generative Ai model and obtains response information.

The input of Step 10 is the prompt data generated in Step 9. The output of Step 10 is response information in natural language that includes content specification information. The server sends the prompt sentence and control parameters to the generative AI model, for example a transformer-based text-generation model accessible via an application programming interface, specifies decoding parameters such as temperature and top-p values, and receives generated text describing recommended content properties, such as genres, mood, tempo, and duration, structured as descriptive sentences.

### Step 11

The Server parses the response information to construct structured content specification information.

The input of Step 11 is the natural-language response information received from the generative AI model. The output of Step 11 is structured content specification information suitable for further processing. The server applies text parsing techniques, such as pattern recognition and keyword extraction guided by the known prompt structure, identifies segments indicating genres, mood descriptors, duration recommendations, and other constraints, and converts the identified elements into a structured representation, such as a set of fields with explicit values or tags.

### Step 12

The server retrieves candidate content based on the content specification information.

The input of Step 12 is the structured content specification information. The output of Step 12 is a set of candidate content items with associated metadata. The server constructs one or more queries that include genre tags, mood descriptors, and duration constraints, transmits the queries to an external content service or an internal storage system, and receives lists of media items that match the queries. The server extracts identifiers, titles, lengths, and other metadata from these lists and stores them locally as candidate content.

### Step 13

The server selects or composes personalized presentation content from the candidate content.

The input of Step 13 is the set of candidate content items, the thought information, and the history information of the user. The output of Step 13l is personalized presentation content, such as a selected playlist or sequence of items. The server calculates a relevance score for each candidate item using a scoring function that reflects alignment with the current mental state, consistency with the content specification information, and diversity relative to previously presented items. The server sorts candidates by their scores, chooses top-ranked items, and composes them into a sequence that satisfies target duration and other constraints, thereby generating personalized presentation content.

### Step 14

The server converts the personalized presentation content into output data and transmits the output data to the terminal.

The input of Step 14 is the personalized presentation content, including selected item identifiers and metadata. The output of Step 14 is transmission of output data to the terminal. The server generates a data structure that includes content identifiers, playback order, descriptive text, and control parameters for presentation, serializes the data structure into a message, and sends the message to the terminal over the network.

### Step 15

The terminal receives the output data and presents the personalized presentation content to the user.

The input of Step 15 is the output data sent by the server. The output of Step 15 is actual presentation of content as visual information and/or auditory information. The terminal deserializes the received data, calls appropriate media playback components based on the content type, connects to content sources or retrieves cached items, starts playback or display according to the prescribed order and parameters, and renders titles, descriptions, and control elements on the display device while outputting audio through the audio output device.

### Step 16

The user interacts with the presented content and provides feedback via the terminal.

The input of Step 16 is the personalized presentation content being displayed or played back by the terminal. The output of Step 16 is user feedback in the form of operation information or evaluation information. The user operates input controls, such as like or dislike buttons, rating sliders, or textual comment fields, to express satisfaction or dissatisfaction. The terminal records these operations together with content identifiers and timestamps and prepares feedback data.

### Step 17

The terminal transmits the feedback data to the server.

The input of Step 17 is the feedback data generated from user interactions. The output of Step 17 is a network message containing the feedback data received by the server. The terminal serializes the feedback data, adds user identifiers and session context, and sends the data to the server using a secure network protocol.

### Step 18

The server stores the feedback information and associates it with the thought information and the personalized presentation content.

The input of Step 18 is the feedback data, together with associated identifiers for thought information and presented content. The output of Step 18 is stored learning data that links input signals, inferred states, selected content, and user evaluations. The server writes the feedback information into a database, associates it with corresponding thought information and content records, and indexes the combined data for later retrieval during training and analysis.

### Step 19

The server updates at least one of the statistical learning model and the generation process of the prompt data based on accumulated learning data.

The input of Step 19 is the accumulated learning data stored across multiple sessions and users. The output of Step 19 is updated model parameters and adjusted prompt-generation rules. The server executes a training or fine-tuning routine for the statistical learning model by computing gradients of a loss function that measures prediction errors and content-selection performance, updates the model's weights via an optimization algorithm, and deploys the updated model for subsequent inference. In parallel, the server analyzes the relationship between specific prompt patterns and resulting feedback, modifies template parameters and rule weights within the prompt-generation module, and thereby refines how future prompt sentences are constructed for the generative AI model.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional brain-computer interface systems and natural language processing systems suffer from several technical limitations when converting neural activity into natural language expressions suitable for real-time communication. First, existing architectures often attempt to directly map low-level brainwave signals to textual outputs using fixed models, which makes the systems highly sensitive to noise, individual variability of neural patterns, and changes in usage context. As a result, the accuracy and stability of the generated text are insufficient for practical communication, particularly in multi-lingual or conversational scenarios.

Second, typical systems do not clearly separate low-level signal interpretation from high-level language generation. Processing pipelines frequently couple brain signal decoding and sentence generation in a monolithic model. This coupling restricts the ability to flexibly change output language, adjust style or politeness level, or incorporate new translation requirements without retraining or substantially modifying the entire model. Consequently, the systems lack scalability and maintainability when applied across different applications, languages, or users.

Third, many existing implementations do not utilize structured, machine-interpretable intermediate representations of user thought content. Without an abstracted “thought pattern” representation, the system cannot reliably reuse or refine internal interpretation results, cannot systematically control prompts given to a generative AI model, and cannot log or analyze long-term correspondences between decoded neural patterns and generated language. This leads to poor adaptability and prevents gradual improvement of decoding precision and language appropriateness for individual users. Fourth, conventional solutions generally do not exploit modern generative AI models via explicit, dynamically constructed prompt sentences that encode both content and output constraints. Instead, they tend to use static prompts or rigid APIs, which limit control over output style, tone, and cross-lingual behavior. Such systems are thus unable to effectively leverage advances in generative AI to improve clarity, naturalness, and context suitability of generated text.

Fifth, typical systems lack a feedback-driven mechanism to adapt system parameters based on accumulated usage history. Without storing and analyzing pairs of “decoded thought pattern” and “generated natural language text,” the systems cannot optimize prompt construction strategies, cannot tune inference conditions of the machine learning model that interprets brain signals, and cannot converge toward user-specific mappings between neural activity and linguistic expressions. This results in persistent mismatch between user intention and output text, and prevents the system from improving over time based on real-world interactions.

Accordingly, there is a need for a technical solution that: (i) performs robust preprocessing and feature extraction on brainwave-based digital signals; (ii) uses a machine learning model to produce an abstracted thought pattern representation; (iii) converts the thought pattern and language requirements into a structured prompt sentence for a generative AI model; (iv) controls the generative AI model to output natural language texts, including multi-lingual outputs; and (v) adaptively updates prompt construction and inference conditions based on stored history. Such a solution should improve the functioning of the underlying computer system itself, by providing a modular architecture and data structures that enhance accuracy, flexibility, and efficiency of brain-to-language conversion, rather than merely automating a mental process.

The specific processing by the specific processing unit 290 of the data processing device 12 in Example 2 is realized by the following means.

The present invention provides a server comprising a processor configured to receive, from a user terminal via a network, structured data including abstracted thought pattern information identified from time-series digital signals representing brain activity of a user, to construct, based on the thought pattern information and on language-related parameters, a prompt sentence to be input to a generative AI model for natural language generation, to transmit the prompt sentence and associated control information to the generative AI model, to obtain from the generative AI model one or more natural language texts in one or more target languages, to store in a storage device at least the thought pattern information and the obtained natural language texts as history information, and to update, based on the stored history information, at least one of configuration conditions for constructing the prompt sentence and inference-related conditions applied by a machine learning model used in the user terminal to generate the thought pattern information. This enables the computer system to implement a modular and adaptive brain-to-language conversion pipeline that improves the technical functioning of the system by enhancing robustness of signal interpretation, controllability of natural language generation through structured prompt sentences, support for multi-lingual and style-constrained outputs, and progressive optimization of mappings between decoded neural signals and natural language expressions for each user.

The term “processor” refers to an electronic data processing unit, including one or more central processing units, graphics processing units, digital signal processors, or other computation circuits, configured to execute instructions and perform arithmetic, logical, control, and input/output operations.

The term “biological signal acquisition apparatus” refers to hardware configured to detect and convert physiological signals of a user, including brain activity signals, into digital data, and to output the digital data to an external device.

The term “brain activity” refers to electrical or related physiological phenomena generated by neural activity in a user's brain, measurable by sensors and representable as time-series data.

The term “time-series digital signals” refers to numerical data sequences that represent values of a measured quantity sampled at successive points in time, where the values have been obtained by converting analog signals into digital form.

The term “signal processing” refers to computational operations performed on time-series digital signals, including filtering, noise reduction, transformation, and feature extraction, to obtain data suitable for subsequent analysis.

The term “frequency-band components” refers to portions of a signal corresponding to specific ranges of frequencies obtained by applying frequency-domain analysis or filtering to the signal.

The term “feature values” refers to numerical descriptors calculated from raw or processed signals, which characterize properties or patterns of the signals and are used as inputs to a machine learning model.

The term “machine learning model” refers to a parametrized computational model, such as a neural network, classifier, or regressor, trained on data to perform inference, including classification or regression, based on input feature values.

The term “inference processing” refers to a computation performed by a machine learning model to produce one or more output values, such as class labels or embeddings, from input data without modifying the model parameters.

The term “thought pattern information” refers to abstracted data representing an internal state, intention, or conceptual content of a user, obtained by interpreting biological signals using a machine learning model.

The term “structured data” refers to data organized according to a predefined schema, such as records, key-value pairs, or hierarchical objects, enabling deterministic parsing and processing by a computer.

The term “network” refers to a communication infrastructure that enables data exchange between devices, including wired or wireless communication paths and associated protocols.

The term “external apparatus” refers to a device or system distinct from a user terminal that communicates via a network, such as a server or remote computing resource.

The term “user terminal” refers to an information processing device operated by or associated with a user, such as a mobile device, wearable device, personal computer, or other client device having communication, display, and optionally audio capabilities.

The term “generative AI model” refers to a computational model based on machine learning that, given input data such as text or parameters, generates new output data, including natural language text, according to learned statistical patterns.

The term “prompt sentence” refers to a sequence of characters or tokens that encodes instructions and contextual information for a generative AI model and is provided as input to control or guide the generation of output data.

The term “natural language text” refers to character strings or token sequences that express information in a human language used for everyday communication.

The term “target output language” refers to a natural language designated as the language in which the generative AI model is requested to generate natural language text.

The term “content description information” refers to a portion of a prompt sentence that describes, in natural language or symbolic form, the meaning, context, or intent corresponding to thought pattern information.

The term “instruction information” refers to data included in or associated with a prompt sentence that specifies constraints or requirements for the output of a generative AI model, such as style, tone, length, format, or expression level.

The term “translation instruction information” refers to data included in or associated with a prompt sentence that specifies one or more target languages and translation-related requirements for the output of a generative AI model.

The term “storage device” refers to a physical or virtual memory component, such as volatile memory, non-volatile memory, or a database system, configured to store data persistently or temporarily.

The term “history information” refers to stored data records that include, for one or more users, past thought pattern information, corresponding generated natural language texts, and associated metadata such as timestamps or parameters.

The term “configuration conditions” refers to rules, parameters, or templates used to determine how a prompt sentence is constructed from thought pattern information and other inputs.

The term “inference-related conditions” refers to parameters, thresholds, or control settings applied to a machine learning model during inference processing, such as confidence thresholds, feature selection rules, or model selection criteria.

The term “presentation” refers to making information perceivable to a user by at least one modality, including visual display of text or auditory reproduction of speech.

The term “speech synthesis process” refers to a computational procedure that converts natural language text into audio data representing spoken utterances.

In one embodiment, the system includes a server, a terminal, and a biological signal acquisition apparatus worn by a user. The biological signal acquisition apparatus includes one or more electrodes placed on the scalp of the user, an analog front-end circuit, an analog-to-digital converter, a microcontroller, and a wireless communication module such as a low-energy wireless transceiver. The biological signal acquisition apparatus measures analog electrical signals corresponding to brain activity, converts the analog signals into time-series digital signals, and transmits the time-series digital signals to the terminal. The terminal includes a processor, a memory, a wireless communication interface, a display, and an audio output unit such as a loudspeaker or headphone jack. The terminal executes software components including a device communication module, a signal processing module, a feature extraction module, and a machine learning inference module. The terminal may execute these modules on a general-purpose operating system such as a mobile operating system and may use a machine learning runtime such as a lightweight neural network inference engine.

The terminal receives time-series digital signals from the biological signal acquisition apparatus via the wireless communication interface. The terminal stores the time-series digital signals in a ring buffer data structure in the memory. The terminal applies digital signal processing operations to the buffered data. For example, the terminal applies a band-pass finite impulse response filter in the range of approximately 0.5 Hz to 40 Hz to remove low-frequency drift and high-frequency noise. The terminal may perform notch filtering at power-line frequencies (e.g., 50 Hz or 60 Hz) to reduce interference. The terminal may compute an artifact index by detecting large transient amplitudes or abnormal high-frequency components, and the terminal may discard or down-weight segments with high artifact indices.

The terminal converts the filtered signals into feature vectors. The terminal divides the time-series digital signals into overlapping windows, for example windows of 1 second with 50% overlap. For each window, the terminal computes a discrete Fourier transform using a fast Fourier transform algorithm and calculates power spectral density values for multiple frequency bands such as delta, theta, alpha, beta, and gamma bands. The terminal may also compute statistical moments such as mean, variance, skewness, and kurtosis of the signal amplitude, and may compute temporal features such as zero-crossing rate and Hjorth parameters. The terminal concatenates these values into a fixed-length feature vector, which becomes an input sample for the machine learning model.

The terminal loads a pre-trained machine learning model from non-volatile memory into the memory. In one embodiment, the machine learning model is a multi-layer neural network that receives the feature vector and outputs a probability distribution over a predefined set of thought pattern classes. The neural network may include an input layer matching the feature dimension, one or more hidden layers such as fully connected layers with rectified linear unit activation functions, and an output layer with a softmax activation function. In another embodiment, the machine learning model includes a temporal convolutional network that applies one-dimensional convolutions over the time dimension of the features to capture temporal dependencies.

The terminal executes the machine learning model in inference mode. The terminal inputs each feature vector to the neural network, performs matrix multiplications and activation function evaluations, and obtains output probabilities for each thought pattern class. The terminal selects the class with the highest probability as a primary thought pattern and records the associated probability as a confidence score. The terminal may also compute an embedding vector from an intermediate hidden layer and use the embedding vector as a continuous representation of the thought pattern.

The terminal constructs a structured data object that represents the identified thought pattern information. The terminal may represent the structured data object as a record including fields such as a user identifier, a thought pattern identifier, a confidence score, an embedding vector, and a timestamp. The terminal may store this structured data object temporarily in the memory and then transmit it to the server via a network using a secure communication protocol, such as a secure transport protocol over an internet protocol network. The server includes a processor, a memory, a network interface, and a storage device such as a database system. The server executes software components including an application programming interface module, a prompt construction module, a generative AI interface module, and a history management module. The server receives the structured data object from the terminal through the application programming interface module and validates that the structured data object includes required fields.

The server converts the thought pattern identifier and related metadata into an intermediate representation suitable for prompt construction. The server maintains a mapping table between thought pattern identifiers and canonical descriptions written in natural language. For example, a thought pattern identifier corresponding to “hunger” may be mapped to a canonical description such as “the user is hungry and wants to eat something soon.” The server retrieves the canonical description from the mapping table based on the received thought pattern identifier.

The server constructs a prompt sentence that serves as an input to a generative AI model. The server combines the canonical description with instruction information specifying language, style, and format. For example, the server may construct a prompt sentence such as:
“Convert the following user thought pattern into a concise natural language sentence in English: the user is hungry and wants to eat something soon.”

In another example, the server may specify politeness and language:
“Using polite Japanese, express the user's thought that they are hungry and would like to request a meal.”

In yet another example, the server may request multiple target languages:
“Convert the following user thought pattern into natural language and provide translations in English, Japanese, and Spanish suitable for everyday conversation during travel: the user is hungry and wants to find a nearby restaurant.”

The server constructs such prompt sentences following templates stored in the memory. The templates may include slots for content description information, desired language, style level, and translation instructions. The server fills the slots with values determined from user settings, application context, and the received thought pattern information.

The server transmits the constructed prompt sentence to a generative AI model that is implemented as a neural network for natural language generation. In one embodiment, the generative AI model is a transformer-based language model that receives a sequence of tokens representing the prompt sentence and outputs a sequence of tokens representing the generated natural language text. The transformer-based language model may include an embedding layer, multiple self-attention layers, feed-forward layers, and a final linear layer that outputs token probabilities over a vocabulary. The server provides the prompt sentence via an application programming interface over a network to a computing environment in which the generative AI model is executed.

The generative AI model processes the prompt sentence token by token. The generative AI model computes attention weights over previously generated tokens and internal states, and applies learned weight matrices to propagate information across layers. The generative AI model outputs a token at each generation step according to a probability distribution, which may be adjusted by decoding strategies such as temperature scaling, top-k sampling, or nucleus sampling. The server configures these decoding parameters as part of control information sent with the prompt sentence.

The server receives from the generative AI model one or more natural language texts in target languages. The server may receive multiple outputs if the prompt sentence specifies multiple languages. The server performs post-processing such as removing leading or trailing whitespace, checking character encodings, and optionally applying a content filter to detect inappropriate or unwanted terms. The server then associates each generated natural language text with the corresponding thought pattern identifier and user identifier.

The server stores records including the thought pattern information and the corresponding generated natural language texts in the storage device. For each record, the server may also store metadata such as the prompt sentence used, decoding parameters, timestamps, and performance metrics. The server uses the history management module to analyze the stored history and to adjust configuration conditions for future prompt sentences. For example, the server may determine that a certain user consistently requires shorter sentences and may adjust the prompt templates to request shorter outputs for that user. The server may also adjust the level of detail or politeness level based on feedback or observed usage patterns.

The server may further use the stored history to provide training data or fine-tuning data for the machine learning model on the terminal. The server can use pairs of feature vectors or embeddings and ground truth labels to refine model weights, using supervised learning. For example, the server can define a loss function such as cross-entropy between model output probabilities and corrected labels and update model weights using an optimization algorithm such as stochastic gradient descent or an adaptive gradient method. The server can then transmit updated model parameters or configuration parameters to the terminal to improve inference performance. This process enables the system to adapt to individual neural patterns and to reduce classification error over time.

The terminal receives updated model parameters or configuration parameters from the server and replaces or augments existing parameters in the machine learning inference module. The terminal may update confidence thresholds, feature normalization statistics, or model architecture selection based on instructions from the server. This allows the terminal to modify its inference conditions in a controlled manner to improve classification accuracy and robustness against noise.

The terminal presents the natural language text received from the server to the user. The terminal displays the natural language text on the display in a user interface element such as a message bubble or caption area. The terminal may also invoke a text-to-speech engine to generate audio waveforms from the text. The text-to-speech engine performs phoneme conversion and prosody synthesis, and the terminal outputs the resulting audio through the audio output unit. Thus, the user can either read or listen to the converted thought content.

In one concrete example, the user thinks “I am hungry and want to find a restaurant near here.” The biological signal acquisition apparatus measures brain activity signals that reflect this mental state, and the terminal processes these signals into feature vectors and then into a thought pattern identifier corresponding to “hunger and restaurant search.” The terminal sends this thought pattern identifier and associated data to the server. The server looks up a canonical description such as “the user is hungry and wants to find a nearby restaurant,” and constructs a prompt sentence such as:
“User's thought pattern indicates that they are hungry and want to find a nearby restaurant. Express this as a concise natural language sentence in English.”

The generative AI model returns a sentence such as “I'm hungry. Could you help me find a nearby restaurant?” The server sends this sentence to the terminal, and the terminal displays and speaks the sentence. The system thereby enables the user to communicate this need without manually composing text or speech.

The described architecture yields technical effects that go beyond mere automation of mental translation. The terminal reduces communication load by transmitting compact thought pattern identifiers and feature representations instead of raw time-series brain data, which can be large. This reduction in data size decreases network bandwidth usage and latency. The separation between the terminal's signal interpretation and the server's language generation allows the server to host more complex generative AI models without overburdening the terminal's processor or memory, leading to improved computational efficiency on resource-constrained devices.

The use of structured data objects and canonical descriptions provides a stable intermediate representation that is reusable across different generative AI models and different language outputs. This intermediate representation allows the server to generate multiple language variants from a single thought pattern interpretation, improving scalability. Furthermore, the feedback mechanism using stored history enables continuous refinement of machine learning models and prompt templates, which improves accuracy and reduces error rates over time. The system improves computer technology by introducing a specific data flow and modular architecture that optimizes processing at each stage. The terminal performs specialized digital signal processing and inference on compressed feature vectors, which reduces computational complexity compared to processing raw time-series data at the server. The server performs high-level language generation and adaptation using learned prompt templates and decoding strategies, which are not achievable by human operators in real time. The combination of these modules produces a synergistic effect: faster overall response time, more accurate mapping between brain activity and language, and more efficient use of network and hardware resources.

In another embodiment, the server may employ multiple generative AI models with different sizes or training data. The server can select an appropriate model based on context, such as choosing a smaller, faster model for low-latency communication or a larger, more accurate model for complex expressions. The server may encode this selection logic in rules that consider factors such as network latency, server load, and past performance metrics. This model selection further optimizes computational resource usage and response quality.

In another embodiment, the terminal may implement different feature extraction pipelines, such as wavelet transform-based analysis or autoregressive modeling of the time-series signals. The terminal may choose a pipeline based on device capability or user-specific characteristics. The server can instruct the terminal to switch pipelines by sending configuration messages. This design enables the system to adapt feature extraction and inference to different hardware and user profiles, thereby achieving consistent performance.

In another embodiment, the system may support multiple communication scenarios such as international exchange, commercial activities, or travel. The server can encode scenario information in the prompt sentence, for example:
“Convert the following user thought pattern into natural language suitable for asking for help at an information counter in an airport.”

By including such scenario-specific instructions, the generative AI model can adjust vocabulary and sentence structure to better match the context. This behavior is controlled by the server's prompt construction logic and does not rely on human authors manually editing each output, thereby leveraging specific computational rules and templates to improve language appropriateness.

By structuring the system as explained above, the server, the terminal, and the user's biological signal acquisition apparatus cooperate to perform data acquisition, signal processing, feature extraction, machine learning inference, structured data exchange, prompt sentence construction, generative AI-based natural language generation, and multimodal presentation. The system uses specific data structures, model architectures, and adaptive control strategies that improve processing speed, precision, and efficiency in converting brain activity into natural language, and therefore constitutes a concrete improvement to computer-implemented communication technology.

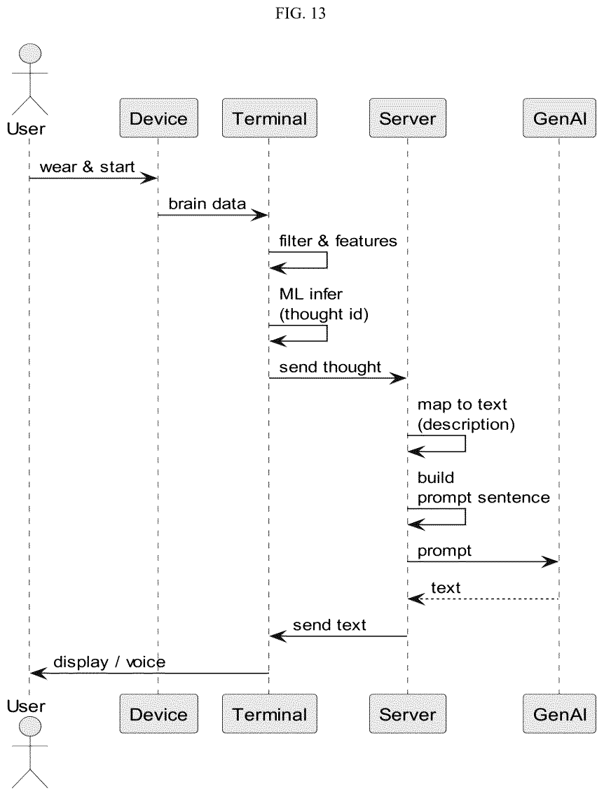

The following describes the processing flow using FIG. 13.

### Step 1

The user wears the biological signal acquisition apparatus on the head and turns on the apparatus. The input is the user's brain activity in the form of analog electrical signals. The output is time-series analog signals available at the input of the apparatus's analog front-end circuit. The apparatus measures potential differences via electrodes, amplifies the signals using an analog amplifier, and forwards the amplified signals to an analog-to-digital converter.

### Step 2

The terminal receives time-series digital signals from the biological signal acquisition apparatus via a wireless communication interface. The input is a stream of digitized brainwave samples transmitted over a wireless protocol. The output is a buffered sequence of time-stamped samples stored in the terminal's memory. The terminal uses a communication module to establish a pairing, decodes received packets, attaches timestamps using a system clock, and writes the samples into a ring buffer data structure.

### Step 3

The terminal performs digital signal processing on the buffered time-series digital signals. The input is a sliding window of samples from the ring buffer. The output is a filtered, artifact-reduced signal window. The terminal applies a band-pass filter to remove components outside a specified frequency range, uses a notch filter to suppress power-line noise, and executes artifact detection algorithms such as threshold-based detection of high-amplitude spikes. The terminal discards or corrects segments flagged as artifacts, thereby producing a cleaned signal.

### Step 4

The terminal extracts feature values from the cleaned signal window. The input is the filtered time-series signal segment. The output is a numerical feature vector representing the segment. The terminal divides the signal into sub-windows, computes fast Fourier transforms, calculates power spectral densities for multiple frequency bands, and aggregates statistics such as mean, variance, and band power ratios. The terminal concatenates these statistics into a fixed-dimensional feature vector and normalizes the vector using pre-stored scaling parameters.

### Step 5

The terminal performs inference using a machine learning model to identify thought pattern information. The input is the normalized feature vector. The output is a thought pattern identifier, a confidence score, and optionally an embedding vector. The terminal forwards the feature vector to a pre-loaded neural network, executes matrix multiplications and non-linear activation functions layer by layer, and obtains a probability distribution over predefined thought pattern classes. The terminal selects the class with the maximum probability as the thought pattern identifier and reads an intermediate hidden layer as the embedding vector.

### Step 6

The terminal constructs structured data including the thought pattern information. The input is the thought pattern identifier, the confidence score, the embedding vector, and a timestamp. The output is a structured record suitable for network transmission. The terminal assigns the user identifier, inserts the thought pattern identifier and associated values into fields of the record, and serializes the record according to a predefined schema. The terminal may include additional metadata such as device identifiers or quality indicators computed from signal processing statistics.

### Step 7

The terminal transmits the structured data to the server via a network. The input is the serialized record containing thought pattern information. The output is a successfully delivered request message stored in the server's input queue. The terminal opens a secure network session, encapsulates the record into a request to a designated endpoint, and performs error checking and retransmission if necessary. The terminal then waits for an acknowledgment or response from the server.

### Step 8

The server receives and validates the structured data from the terminal. The input is the incoming request containing the serialized record. The output is a parsed internal representation of the thought pattern information. The server decodes the request, authenticates the terminal using stored credentials, and parses the serialized content into internal fields. The server checks the presence and format of required fields, discards invalid records, and forwards valid records to subsequent processing modules.

### Step 9

The server maps the thought pattern identifier to canonical content description information. The input is the thought pattern identifier and related metadata. The output is a canonical natural language description of the user's internal state. The server accesses a mapping table or rule set, searches for an entry corresponding to the thought pattern identifier, and retrieves a base description such as “the user is hungry and wants to eat something soon.” If metadata indicates additional context, the server modifies the base description accordingly, for example by appending location-related information.

### Step 10

The server constructs a prompt sentence for a generative AI model based on the canonical description and language-related parameters. The input is the canonical description, the user's language preferences, style settings, and any translation requirements. The output is a complete prompt sentence in text form. The server selects a template that encodes instructions, such as “Convert the following user thought pattern into a concise natural language sentence in English: [description].” The server substitutes the canonical description into the template and adds directives about tone and output length, producing prompt sentences such as:
“Convert the following user thought pattern into a concise natural language sentence in English: the user is hungry and wants to eat something soon.” or
“Using polite Japanese, express the user's thought that they are hungry and would like to request a meal.”

### Step 11

The server sends the prompt sentence to the generative AI model and requests natural language generation. The input is the constructed prompt sentence and optional control parameters such as decoding temperature or maximum token length. The output is one or more generated natural language texts. The server transmits the prompt sentence to a language generation endpoint, specifies generation parameters, and waits for the response. The generative AI model processes the tokens of the prompt, computes self-attention and feed-forward transformations across multiple layers, and returns sequences of tokens representing sentences in the requested languages.

### Step 12

The server post-processes the generated natural language texts. The input is the raw text output from the generative AI model. The output is cleaned, formatted natural language text associated with the original thought pattern. The server trims extraneous whitespace, ensures that encodings conform to expected standards, and may apply content filters based on a rule set to remove or flag unsuitable expressions. The server then packages the final texts into a structured response that includes the thought pattern identifier and any language codes indicating the target languages.

### Step 13

The server stores history information for adaptive optimization. The input is the thought pattern information, the prompt sentence used, and the generated natural language texts. The output is a set of persistent records in a storage device. The server writes entries that link each thought pattern identifier and embedding vector to the corresponding generated texts, along with timestamps and configuration parameters. The server later analyzes these records to adjust prompt templates and model-related thresholds, using algorithms that compute statistics such as error frequencies, user-specific preferences, and distribution of output lengths.

### Step 14

The server transmits the generated natural language texts to the terminal. The input is the structured response that includes one or more natural language sentences. The output is a network response delivered to the terminal. The server encodes the texts and associated identifiers into a response message, sends the message over the established secure session, and records transmission status for monitoring or retry control.

### Step 15

The terminal receives and parses the response from the server. The input is the network response containing the generated natural language texts. The output is internal data structures holding the texts for presentation. The terminal reads the response, checks integrity and authenticity, and extracts fields such as primary language text and any additional translations. The terminal stores these values in memory for subsequent display and audio output.

### Step 16

The terminal presents the natural language text to the user. The input is the generated natural language text in one or more languages. The output is a visual and/or auditory presentation perceivable by the user. The terminal renders the text on the display using a graphical user interface component, and, when enabled, sends the text to a text-to-speech engine. The terminal receives audio samples from the text-to-speech engine, configures volume and playback parameters, and plays the audio through a speaker or headphones, thereby expressing the user's decoded thought as spoken language.

### Step 17

The server and the terminal cooperatively update configuration and model parameters based on accumulated history. The input is history information stored on the server and performance indicators such as user feedback or inferred accuracy metrics. The output is updated prompt construction rules on the server and updated inference conditions or model parameters on the terminal. The server analyzes patterns in stored records, identifies cases where outputs required correction or adjustment, and computes revised parameters, such as modified template wording or adjusted confidence thresholds. The server then sends configuration updates or new model weights to the terminal. The terminal applies those updates by replacing old parameters, adjusting feature normalization statistics, and, if necessary, switching to a different model variant, thereby improving future conversions from brain activity to natural language.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional human-computer interaction systems that rely on manual input devices, such as keyboards, pointing devices, and touch interfaces, or on conventional speech interfaces, introduce substantial latency and cognitive overhead when users attempt to express complex intentions or emotional states. In particular, existing systems typically require the user to explicitly formulate text or speech commands and do not directly utilize biological signals such as brain activity for intent determination. As a result, such systems are unable to provide real-time, fine-grained adaptation of system behavior based on continuously changing user thoughts and emotions.

Furthermore, typical signal-processing pipelines for biological signals, including brainwave signals, are designed for offline analysis or limited classification tasks and are not integrated with generative models that can dynamically synthesize natural-language outputs or machine-interpretable control data. In these architectures, the biological-signal processing, the natural-language generation, and the downstream device control are implemented as separate, loosely coupled components. This fragmented design leads to increased processing latency, duplicated computation, and inconsistent state between modules, thereby limiting the responsiveness and robustness of the system.

In addition, existing dialog systems and search systems usually operate on explicit textual or spoken input that has already been linguistically formed by the user. These systems do not incorporate a unified mechanism that (i) acquires the user's biological signals as digital signals, (ii) extracts feature amounts to specify user thought content and emotional state, (iii) automatically constructs prompt sentences for a generative model, and (iv) converts the generative model's output into both user-facing content and machine-control data. Consequently, current systems cannot efficiently exploit low-level biological signals to improve the quality of generated responses, to adapt the prosody of synthesized speech, or to adjust control strategies for operation apparatuses in real time.

Moreover, many existing multilingual and translation systems treat language conversion as an isolated task. They do not integrate language selection, output-format selection, and emotion-dependent adaptation into the prompt generation and response-generation pipeline. This leads to additional application-level logic, greater complexity in client software, and greater susceptibility to implementation errors, which in turn degrade overall system performance and developer productivity.

Accordingly, there is a need for an improved computer-implemented system and server architecture that tightly integrates biological-signal acquisition, signal processing, intent classification, emotion estimation, prompt-sentence generation for a generative model, and downstream text-to-speech and device-control functions. Such an architecture should reduce latency, decrease the amount of client-side processing, and improve the accuracy and contextual appropriateness of generated natural-language outputs, audio outputs, and control outputs. There is a further need for a unified mechanism to automatically configure input language, output language, and output format in prompt sentences, thereby improving the flexibility, scalability, and maintainability of multilingual, multi-modal human-computer interfaces.

The specific processing by the specific processing unit 290 of the data processing device 12 in Application Example 2 is realized by the following means.

The present invention provides a server comprising a processor configured to acquire a biological signal of a user and obtain the biological signal as a digital signal, to perform signal processing on the obtained digital signal to extract a feature amount and, on the basis of the feature amount, to specify thought content of the user and to estimate an emotional state of the user, to generate a prompt sentence on the basis of the specified thought content and the estimated emotional state, the prompt sentence instructing a generative data generation apparatus to generate natural language, to translate or summarize natural language, or to generate control data for an operation apparatus, to input the prompt sentence into the generative data generation apparatus and obtain, from the generative data generation apparatus, a generation result as natural-language data or as structured data, to generate audio data by using a speech synthesis apparatus on the basis of the obtained generation result and to adjust an audio output characteristic of the audio data in accordance with the estimated emotional state, and to control an information processing apparatus or an operation apparatus in accordance with the obtained generation result or control data generated on the basis of the obtained generation result. This enables an integrated, low-latency, and context-aware improvement of computer technology, in which the server internally unifies biological-signal analysis, generative-model prompting, natural-language and control-data generation, emotion-adaptive speech synthesis, and device control, thereby enhancing the efficiency, responsiveness, and robustness of human-computer interaction and reducing the computational and implementation burden on client devices.

The term “biological signal” refers to an electrical or physiological signal generated by a living body, including but not limited to a brainwave signal, a cardiac signal, a muscular signal, or a skin-conductance signal, which can be measured by a sensor and converted into a digital signal for processing by a computing apparatus.

The term “digital signal” refers to a representation of an analog biological signal in a discrete, quantized form suitable for storage, transmission, and processing by a computing apparatus.

The term “signal processing” refers to a sequence of computational operations applied to a digital signal, including at least one of filtering, noise reduction, normalization, segmentation, feature extraction, and transformation, to obtain information useful for subsequent analysis by a computing apparatus.

The term “feature amount” refers to a numerical value or a set of numerical values derived from a digital signal through signal processing, which characterizes the temporal, spectral, statistical, or structural properties of the signal and is used as input to a classification, estimation, or generation process executed by a computing apparatus.

The term “thought content” refers to an estimated representation of an intention, command, query, preference, or other cognitive state of a user, inferred from a biological signal or other input data by a computing apparatus.

The term “emotional state” refers to an estimated representation of an affective condition of a user, such as joy, sadness, anger, stress, relaxation, or neutrality, inferred from a biological signal, textual data, audio data, or other input data by a computing apparatus.

The term “prompt sentence” refers to a text string or structured textual data that specifies, in natural language or a markup format, an instruction to a generative data generation apparatus regarding a target task, a desired output content, a language, or an output format.

The term “generative data generation apparatus” refers to a hardware or software component, including a model implemented by machine learning, that receives a prompt sentence as input and generates, as output, natural-language data or structured data by performing at least one of text generation, translation, summarization, or data-format transformation.

The term “natural-language data” refers to text expressed in a human language, such as a sentence, a phrase, a paragraph, or a dialog turn, that is interpretable by a human user and generated or processed by a computing apparatus.

The term “structured data” refers to data organized according to a predefined schema or format, such as a key-value structure, a tree structure, a table structure, or a serialized markup or notation, which is suitable for direct processing by an information processing apparatus or an operation apparatus.

The term “speech synthesis apparatus” refers to a hardware or software component that converts natural-language data or phonetic data into audio data representing speech, including control over at least one of pitch, speed, volume, timbre, and prosodic pattern.

The term “audio output characteristic” refers to a property of audio data or synthesized speech, including at least one of pitch, speaking rate, volume, timbre, prosody, pause pattern, or emphasis pattern, which can be adjusted by a computing apparatus.

The term “information processing apparatus” refers to an electronic device that executes an information-processing function, such as a server, a client terminal, a computing node, a display device, or a communication device, under control of a processor.

The term “operation apparatus” refers to an apparatus that performs a physical action or a mechanical operation under control of control data, including but not limited to a robot, an actuator system, a manufacturing machine, a vehicle subsystem, or a household appliance.

The term “control data” refers to data describing an operation to be executed by an operation apparatus, including at least one of an action type, a target object, a parameter value, a timing value, or a trajectory specification, which is interpretable by a control interface of the operation apparatus.

The term “search processing apparatus” refers to a hardware or software component that receives a search request and retrieves associated information from at least one of a database, an index, or an external information source, and outputs a search result to a requester.

The term “display apparatus” refers to a device or component that presents visual information to a user, including but not limited to a flat-panel display, a head-mounted display, a projection display, or a wearable visual display.

The term “input language” refers to a human language in which an input text or a user instruction is expressed and which is specified to a generative data generation apparatus as part of a prompt sentence.

The term “output language” refers to a human language in which a generative data generation apparatus is requested, by a prompt sentence, to generate natural-language data.

The term “output format” refers to a structural or syntactic specification indicating how a generative data generation apparatus should format output data, including at least one of plain text, a list, a table, a markup representation, or a control-oriented structured data representation.

The term “control-oriented structured data” refers to structured data that encodes an operation command or configuration information for an operation apparatus in a machine-interpretable format, such as a key-value document, a message object, or a protocol-specific control frame.

In one embodiment, a server, a terminal, and at least one operation apparatus together implement the system. The server includes at least one processor, a memory, a network interface, and a non-transitory storage medium storing program instructions. The terminal includes an input interface, an audio output interface, and optionally a biological-signal sensor interface and a display apparatus. The operation apparatus includes a control interface capable of receiving control data from the server.

The server executes a program that is configured to acquire a biological signal of a user, to perform numerical processing on the signal, to classify thought content and an emotional state, to generate a prompt sentence for a generative AI model, to obtain natural-language data or structured data from the model, and to convert such data into control data and audio data for controlling the operation apparatus and for outputting speech through the terminal. The program is stored in the non-transitory storage medium and executed by the processor in response to system initialization or an external request from the terminal.

In one hardware configuration, the user wears an electroencephalography (EEG) device on the head. The EEG device includes multiple electrodes, an analog front-end circuit, and an analog-to-digital converter (ADC). The terminal connects to the EEG device via a wireless link such as Bluetooth Low Energy or via a wired link such as a universal serial bus. The terminal executes driver software supplied with the EEG device and periodically acquires raw EEG samples from the ADC as signed integer values. The terminal packs these values with timestamps into a data structure, such as a fixed-length frame containing channel identifiers and sampling period information, and transmits the frames to the server through a network interface using a protocol such as Hypertext Transfer Protocol over Transport Layer Security or a persistent bidirectional channel such as a WebSocket connection.

The server receives the frames and stores them in a buffer region of the memory. The server organizes the samples into a multidimensional array that represents a sliding time window across multiple channels. The server applies digital signal processing operations implemented with numerical libraries such as array-based linear algebra modules and fast Fourier transform modules. The server applies at least a band-pass filter that passes frequencies in a range suited for brainwave analysis (for example, 0.5 to 40 Hz) and a notch filter centered around the power-line frequency (for example, 50 or 60 Hz). The server normalizes each channel by subtracting the channel-wise mean and dividing by the channel-wise standard deviation computed over a calibration period.

The server extracts feature amounts from the filtered and normalized data. The server computes a power spectrum using a fast Fourier transform for each channel and aggregates spectral power in predefined sub-bands corresponding to delta, theta, alpha, beta, and gamma ranges. The server also computes time-domain features such as mean amplitude, variance, and higher-order statistics for each channel. The server concatenates the sub-band powers and time-domain statistics into a feature vector for each time window. The server stores each feature vector in a structured data object that also includes metadata such as window index and channel configuration. The server classifies the thought content of the user by inputting the feature vector into a pre-trained neural network or other classifier. In one embodiment, the server uses a feed-forward neural network with multiple hidden layers. Each hidden layer includes a linear transformation and a non-linear activation function such as a rectified linear unit. The output layer produces a probability distribution over a set of intent classes, such as “assemble_next_part”, “proceed_to_next_step”, “request_information”, “request_music”, or “request_rest”. The server determines the predicted intent by selecting the class having the highest probability, subject to a predetermined confidence threshold. The server trains this neural network offline by supervised learning. During training, the server uses recorded EEG feature vectors paired with labels representing known user intentions. The server uses a loss function such as cross-entropy and updates the weights through gradient-based optimization such as stochastic gradient descent or an adaptive variant. The server may apply data augmentation techniques such as adding small Gaussian noise to feature vectors or randomly shifting the time windows to improve robustness.

The server also estimates the emotional state of the user by using an emotion-classification model. In one embodiment, the server shares lower-layer parameters between the intent classifier and the emotion classifier and adds a separate output head for emotion labels such as “joy”, “neutral”, “stress”, and “fatigue”. The server computes an emotion output vector by applying a softmax function over the emotion logits and selects the emotion label with the highest probability. The server may also compute continuous values such as valence and arousal by using regression outputs in the same network. The server periodically recalibrates baseline emotion thresholds using data collected while the user is at rest. The server constructs a prompt sentence for a generative AI model using the predicted intent and the estimated emotional state. The server stores a plurality of template strings in the memory. Each template string describes a particular target task and output format. The server selects a template according to the intent classification result and fills in variable portions of the template with the detected thought content, emotion label, input language, and desired output language. Examples of such prompt sentences include:
“User's thought pattern has been detected as: ‘Assemble the next part.’ Convert this into a clear and concise instruction suitable for controlling an industrial machine.”
“User's thought is: ‘Proceed to the next step.’ The emotion engine indicates stress. Generate a calm and reassuring sentence that confirms the action and suggests a slower, safer pace.”
“The user's thought is: ‘Looking for a new smartphone.’. Translate this into natural English and generate a short explanation that can be used to assist a customer.”
“The user feels stressed according to EEG-based emotion analysis. Recommend several types of relaxing music and briefly explain why they are effective.”

In another embodiment, the user does not use an EEG device but inputs text directly through the terminal. The terminal displays a text input field and receives a natural-language sentence such as “I want to drink coffee” or “I am very happy today.” The terminal transmits this text to the server. The server analyzes the text with a sentiment-analysis module that uses a neural network, such as a bidirectional recurrent network or a transformer encoder trained on labeled sentiment data, to detect an emotional state. The server then constructs a prompt sentence that combines the user's text and the detected sentiment, such as: “The user wrote: ‘I am very happy today.’ Generate a short, positive response that celebrates this feeling.”

The server sends the generated prompt sentence to an external or internal generative AI model. In one embodiment, the server accesses a remote generative AI model via a network API. The server formats an API request body containing the prompt sentence, a model identifier, and generation parameters such as a temperature value, a maximum output length, and a top-probability truncation parameter. The server transmits the request through the network interface and receives a response containing generated text. The generative AI model may be a transformer-based language model having multiple layers of self-attention and feed-forward sub-layers, trained on a large corpus of text. The server does not rely solely on a generic text generation function but constrains the behavior of the model through carefully structured prompt sentences that encode both technical context (device control vs. dialogue) and emotion-dependent requirements (calm tone, reassuring message, etc.).

The server parses the response from the generative AI model. In a machine-control scenario, the server may instruct the generative AI model, through the prompt sentence, to output a specific structured format such as a list of parameterized actions in plain text. The server then applies rule-based parsing to convert the plain-text description into structured control data. For example, if the generated text is “Move the arm to the next component and assemble it onto the frame,” the server may map this to control data that specifies an action type “move”, a target “next_component”, and a subsequent action type “assemble”. The server stores the control data in a control-oriented structured data object and sends it via a control protocol to the operation apparatus.

In one embodiment, the operation apparatus is an industrial robot, a manufacturing machine, or another mechanical system. The server communicates with the operation apparatus via an industrial communication protocol and transmits control frames derived from the control data. The operation apparatus executes the commands, thereby physically moving mechanical components or performing assembly operations. Because the server uses the EEG-based classification to determine the user's intent and emotion and uses the generative AI model to refine and expand the instruction, the resulting control data is more contextually appropriate and can adjust machine behavior, such as operation speed or safety margin, according to the user's emotional state. For example, when the emotion classification indicates stress, the server reduces the step size or speed parameter before sending it to the operation apparatus.

The server also generates audio data for user feedback. The server provides the generated natural-language text to a speech synthesis apparatus. The speech synthesis apparatus may be a cloud-based text-to-speech service or an on-premise synthesis engine that converts text into waveform samples. The server configures synthesis parameters such as pitch, speaking rate, and prosodic emphasis in accordance with the estimated emotional state. For a stressed state, the server selects a slower speaking rate and a lower pitch to produce a calming effect, whereas for a joyful state, the server selects a slightly higher pitch and more energetic prosody. The server then transmits the resulting audio data to the terminal. The terminal outputs the audio through speakers or a headset, enabling the user to hear confirmations, explanations, or recommendations in real time.

The server can also control an information processing apparatus such as a search engine. The server may use the generative AI model's output as a refined search query and send this query to a search processing apparatus. The search processing apparatus returns search results. The server formats these results and optionally summarizes them by sending another prompt sentence to the generative AI model, requesting a concise explanation. The server then outputs the summarized results to the user through both the speech synthesis apparatus and the terminal's display apparatus. This integration reduces the amount of data that must be transmitted to the terminal and decreases cognitive load on the user, thereby improving the responsiveness and efficiency of the overall system.

The technical effect of this architecture is that the server implements a specialized, low-latency data flow that combines biological-signal analysis, neural classification, generative prompting, and device control in a unified pipeline. By performing signal processing and classification on server-side hardware using optimized numerical libraries and neural networks, the system reduces the amount of raw biological data transmitted across the network, which lowers communication load and latency. By generating prompt sentences that embed structured technical constraints and emotional context, the server reduces the need for complex business-logic code on the client side and increases the accuracy and relevance of the generative AI model outputs. The server also improves computational efficiency by reusing feature vectors for both intent and emotion classification and by sharing lower-layer parameters between multiple output heads of a neural network.

The server improves computer technology itself by introducing a specific, non-conventional arrangement of modules and data structures. The server does not merely automate a human task of composing sentences or machine commands. Instead, the server performs a sequence of numerical operations that a human could not perform at comparable speed or scale, such as real-time spectral analysis of multi-channel EEG signals, classification through high-dimensional feature spaces, and dynamic reconfiguration of generative model behavior through programmatically composed prompt sentences. This arrangement reduces overall processing time from signal acquisition to machine actuation, reduces misclassification errors through joint optimization of shared network parameters, and improves robustness to noise by using data augmentation and normalization techniques.

In another embodiment, the server employs a transformer-based encoder to process feature sequences derived from time-ordered windows of EEG data. The server divides the EEG time series into overlapping segments, projects each feature vector into an embedding space, and feeds the resulting sequence of embeddings into stacked self-attention layers. The transformer encoder captures long-range dependencies and temporal patterns that are not easily captured by simple feed-forward networks. The server trains this encoder with a multi-task loss that combines an intent classification term and an emotion classification term, thereby enabling the network to share information between tasks and improving generalization. The server then uses the encoder outputs to construct richer prompt sentences that reference temporal patterns, such as “The user has been continuously stressed for the last minute.”

In a further embodiment, the server adjusts its internal algorithms based on runtime statistics. The server monitors accuracy metrics for intent classification and emotion classification using periodic labels collected during supervised calibration sessions with the user. When the server detects drift in classification performance, the server updates model weights using incremental learning techniques on newly collected data, while maintaining constraints on maximum deviation from the original model. The server may also adjust threshold values for accepting or rejecting low-confidence predictions. This dynamic adaptation improves long-term performance and reduces misinterpretation of user intent.

The terminal may also include a wearable visual display such as a head-mounted display or smart glasses. The server formats the emotional state and key actionable hints into short messages and sends them to the terminal, which overlays them in the user's field of view. For example, when a second user acts as an operator or assistant, the terminal may display a message such as “The other user appears anxious. Provide reassurance,” enabling more appropriate human-human interaction mediated by the system.

Alternative embodiments may vary the type of biological signal used. The server may process cardiac signals or muscle activity in addition to or instead of EEG, using feature extraction suited to those modalities, such as heart rate variability features or muscle activation envelopes. The server may also vary the machine-learning architecture, using convolutional neural networks for processing spatially arranged sensor channels or hybrid architectures combining convolutional and recurrent units. The core concept remains that the server acquires biological signals, extracts feature amounts, classifies thought content and emotional state, generates prompt sentences for a generative AI model, and converts the model outputs into natural-language data, audio data, and control data in a technically integrated manner. Because the system is implemented as a set of cooperating modules executed by the server and terminal, and because the biological-signal processing, generative prompting, and control decisions are all realized through specific computational algorithms and data structures on electronic hardware, the invention provides an improvement to the functioning of the computer system itself. The server reduces redundant communication, optimizes internal model usage, and produces control outputs and audio outputs that are technically adapted to the user's biological state, thereby achieving improved processing speed, improved accuracy of intent and emotion recognition, reduced error rates in operation apparatus control, and more efficient data management within the computing environment.

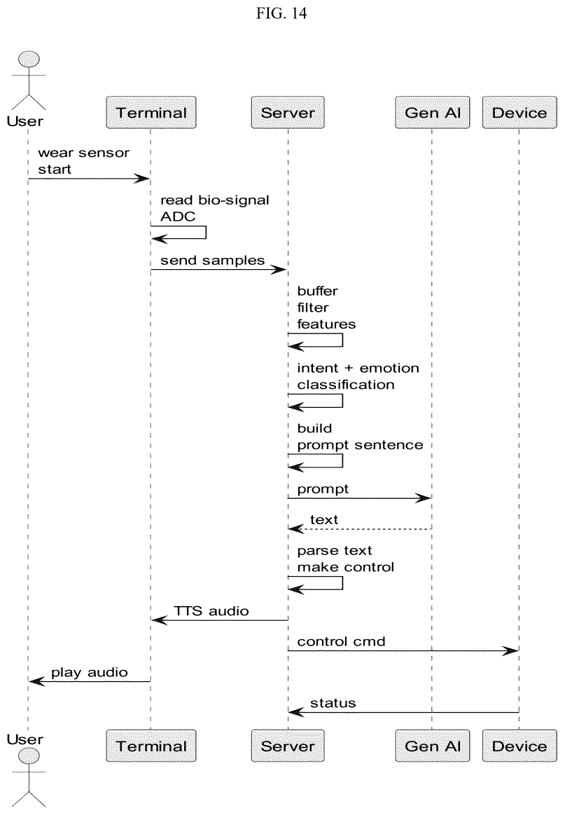

The following describes the processing flow using FIG. 14.

### Step 1

User wears a biological-signal sensor device on the body, such as an EEG headset, and starts an interaction session. I

nput: user's analog biological signal (e.g., brainwave signal).

Output: analog electrical signals at the sensor electrodes.

User positions the electrodes according to a predefined layout and powers on the device. Based on the physical contact with the skin, the sensor generates continuous analog voltage signals corresponding to the user's biological activity.

### Step 2

Terminal acquires the analog biological signal from the sensor and converts it into a digital signal.

Input: analog electrical signals from the sensor's analog front-end.

Output: digital samples (e.g., integer values per channel with timestamps).

Terminal uses a driver program to communicate with the sensor's analog-to-digital converter, samples each channel at a fixed sampling rate, and packs the raw integer samples with timestamps into data frames. Terminal thus performs analog-to-digital conversion and basic framing of the data.

### Step 3

Terminal transmits the digital biological-signal data to the server via a communication network. Input: framed digital samples (multi-channel, time-stamped). Output: network packets containing framed digital data. Terminal encapsulates each frame into a message format (for example, JSON or binary) and sends the messages over a secure channel such as HTTPS or WebSocket to a predefined server endpoint. Terminal may compress the data or batch multiple frames together to reduce communication overhead.

### Step 4

Server receives the digital biological-signal data and buffers it in a temporal window.

Input: network packets containing framed digital biological-signal data.

Output: buffered multi-dimensional array representing a time window of samples.

Server decodes the received messages, checks sequence numbers and timestamps, and writes the samples into a ring buffer. Server then extracts a fixed-length window (for example, several seconds) of data across all channels and stores it as a numeric array for further processing.

### Step 5

Server performs signal processing on the buffered data to reduce noise and normalize the signals.

Input: multi-channel numeric array for a time window.

Output: filtered and normalized numeric array.

Server applies a band-pass filter to remove very low-frequency drift and high-frequency noise, and applies a notch filter at the power-line frequency. Server then computes channel-wise mean and standard deviation over a calibration period and uses these values to normalize each channel within the current window, thereby generating a cleaned and normalized representation of the biological signal.

### Step 6

Server extracts feature amounts from the processed data for machine-learning analysis.

Input: filtered and normalized numeric array.

Output: feature vector representing statistical and spectral characteristics.

Server computes a fast Fourier transform for each channel, calculates power in predefined frequency bands, and computes time-domain metrics such as mean, variance, and higher-order statistics. Server concatenates these values into a single feature vector, optionally adds metadata such as window index or sensor configuration, and stores the feature vector in memory.

### Step 7

Server classifies the thought content of the user based on the feature vector.

Input: feature vector derived from the biological signal.

Output: predicted intent label and confidence value.

Server inputs the feature vector into a pre-trained neural network classifier, performs a forward pass through multiple layers with nonlinear activations, and obtains a probability distribution over possible intent classes. Server selects the class with the highest probability as the predicted thought content, provided that the probability exceeds a predefined threshold, and outputs both the label and the confidence score.

### Step 8

Server estimates the emotional state of the user from the same feature vector.

Input: feature vector derived from the biological signal.

Output: predicted emotional state label and optional continuous emotion metrics. Server feeds the feature vector into an emotion-classification model, which may share lower-level parameters with the intent classifier, and computes emotion logits for categories such as “joy”, “stress”, and “neutral”. Server applies a softmax function to obtain probabilities, selects the most likely emotion label, and optionally computes continuous values such as valence and arousal. Server records the emotional state for use in later processing and logging.

### Step 9

Server constructs an internal representation of the user state combining thought content and emotional state.

Input: predicted intent label, confidence value, emotional state label, and optional emotion metrics.

Output: structured user-state object.

Server creates a data structure such as a key-value object that stores the intent, emotion, current language settings, and session context. Server may include history information, such as prior intents and emotions, and flags indicating whether the current prediction is stable or needs confirmation. This structured user-state object serves as the basis for generating a prompt sentence.

### Step 10

Server generates a prompt sentence for a generative AI model using the user-state object.

Input: structured user-state object containing intent, emotion, and language context.

Output: prompt sentence in text form.

Server selects a template string according to the intent category, replaces placeholders with natural-language expressions of the detected thought and emotion, and adds instructions regarding target language and output format. For example, Server generates a prompt sentence such as:
“User's thought pattern has been detected as: ‘Assemble the next part.’ Convert this into a clear and concise instruction suitable for controlling an industrial machine.” or
“User's thought is: ‘Proceed to the next step.’ The emotion analysis indicates stress. Generate a calm and reassuring sentence that confirms the action and suggests a slower, safer pace.”

### Step 11

Server sends the prompt sentence to a generative AI model and obtains generated output.

Input: prompt sentence specifying the task, context, and desired output characteristics.

Output: generated natural-language text or structured description.

Server formats the prompt sentence into an API request, sets parameters such as temperature and maximum length, and transmits the request to the generative AI model via a network interface or an internal model interface. Server receives the response, extracts the main generated text from the response structure, and stores it for further processing.

### Step 12

Server converts the generated text into control-oriented structured data when machine control is required.

Input: generated natural-language text describing an action or sequence of actions.

Output: structured control data for an operation apparatus.

Server applies parsing rules or pattern-matching logic to the text, identifies action verbs, targets, and parameters, and maps them to predefined control fields such as action type, target object, position, speed, and safety margin. Server packages these fields into a structured control message that conforms to the protocol of the operation apparatus.

### Step 13

Server generates audio data from the generated text using a speech synthesis apparatus.

Input: generated natural-language text and estimated emotional state.

Output: audio data representing synthesized speech.

Server passes the text to a text-to-speech engine, selects a voice profile, and configures synthesis parameters such as pitch and speaking rate according to the emotional state. For example, if the emotional state indicates stress, Server lowers the speaking rate and reduces pitch variations to produce a calmer tone. Server triggers the synthesis process and receives an audio file or audio stream as output.

### Step 14

Server transmits the audio data and any relevant text to the terminal for presentation to the user.

Input: audio data and, optionally, text transcript and emotion metadata.

Output: network packets delivering audio and metadata to the terminal.

Server encapsulates the audio data and metadata into messages, sends them via a communication protocol to the terminal, and may include identifiers linking the audio to the corresponding user-state object or operation apparatus command. Server ensures correct ordering and integrity through sequence numbers or checksums.

### Step 15

Terminal plays back the audio data and optionally displays text and emotion information.

Input: audio data and metadata received from the server.

Output: audible speech and visual information presented to the user.

Terminal decodes the audio data, routes the resulting audio samples to the operating system's sound subsystem, and outputs the sound through speakers or a headset. Terminal may simultaneously display the text transcript of the generated response and a short indication of the emotional state, allowing User to confirm the content and tone of the system's feedback.

### Step 16

Server sends structured control data to the operation apparatus and monitors its execution.

Input: structured control data describing desired operations.

Output: control commands transmitted to the operation apparatus and status information returned.

Server maps the structured control data to low-level control frames or function calls according to the operation apparatus protocol, transmits them via an industrial communication interface, and receives status responses indicating completion, errors, or intermediate states. Server logs the status and may adjust subsequent commands based on success or failure information.

### Step 17

User optionally provides additional explicit input via the terminal to refine or override system behavior.

Input: explicit user input such as text commands, button presses, or menu selections.

Output: updated instructions or constraints received by the server.

User may type text such as “slow down the machine” or “repeat the instruction” on the terminal. Terminal sends this input to Server, which integrates it with the current user-state object, potentially updates the prompt sentence, and re-invokes the generative AI model to produce refined text, control data, and audio feedback.

### Step 18

Server logs data and updates models or thresholds based on performance metrics.

Input: feature vectors, predicted intents, emotional states, generated outputs, and operation apparatus feedback.

Output: stored log records and updated model parameters or decision thresholds.

Server writes the collected information into a persistent storage system, associates predictions with actual outcomes where available, and computes metrics such as accuracy and latency. Server may periodically run an update routine that adjusts intent-classification thresholds, refines emotion-classification boundaries, or performs incremental training on stored data to improve future predictions.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

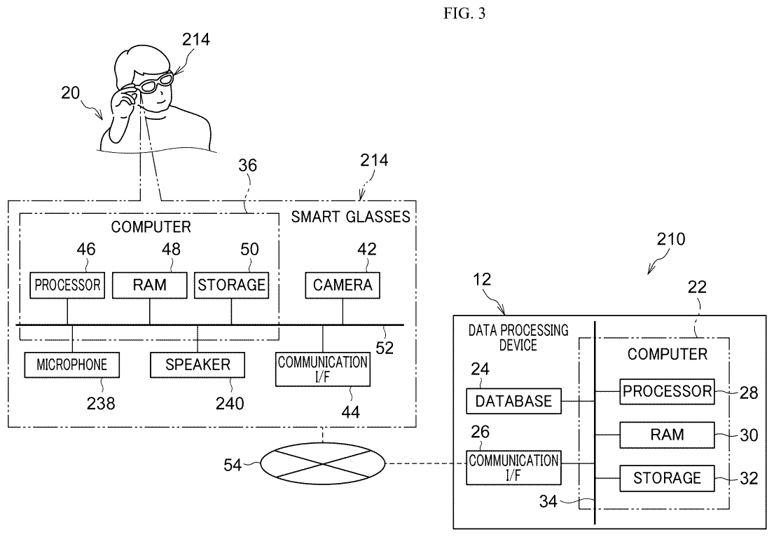

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

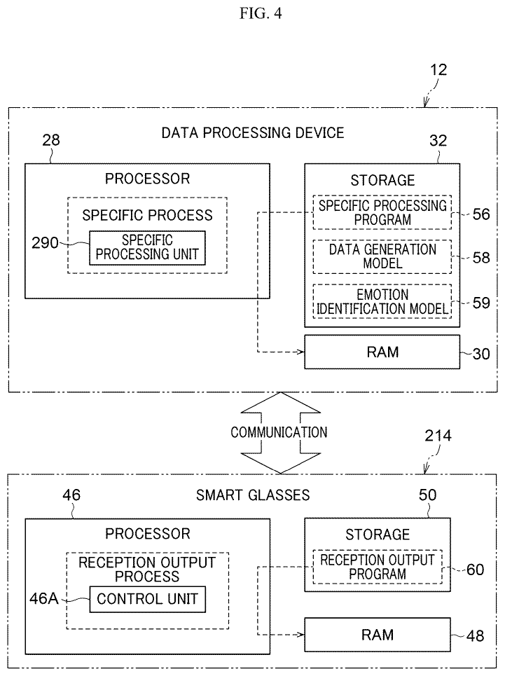

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https: //openai. com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

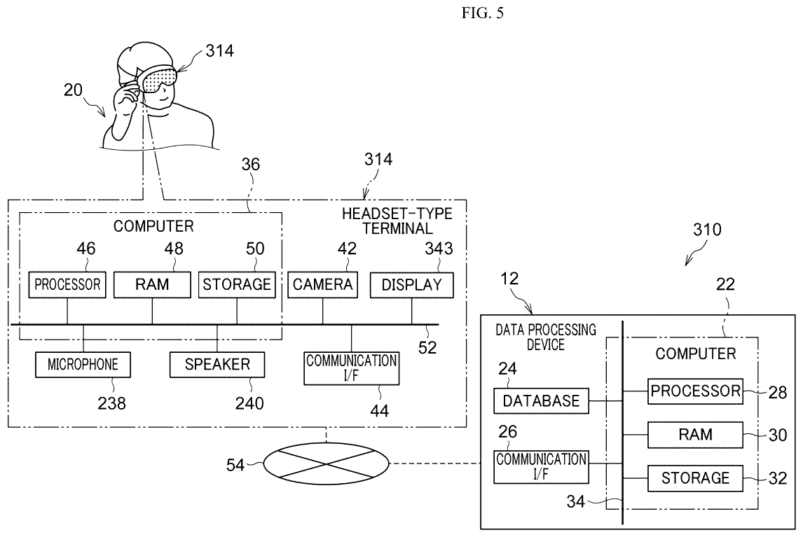

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

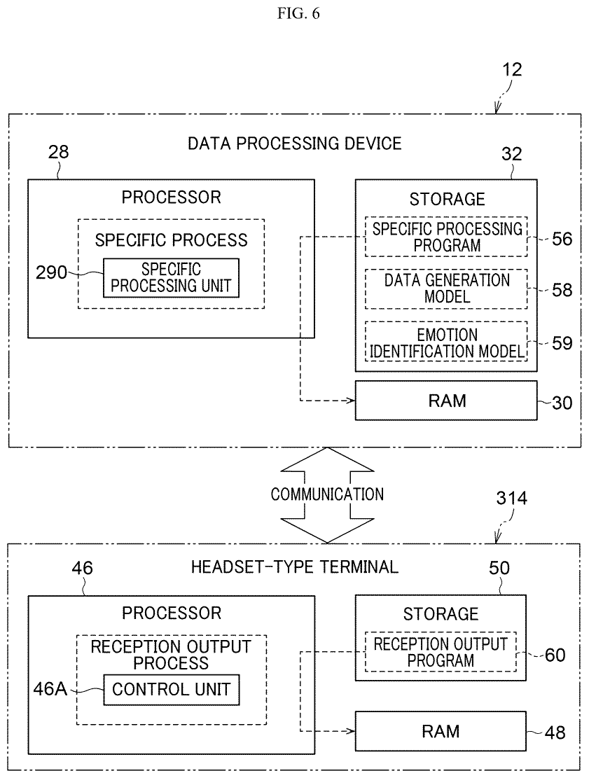

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

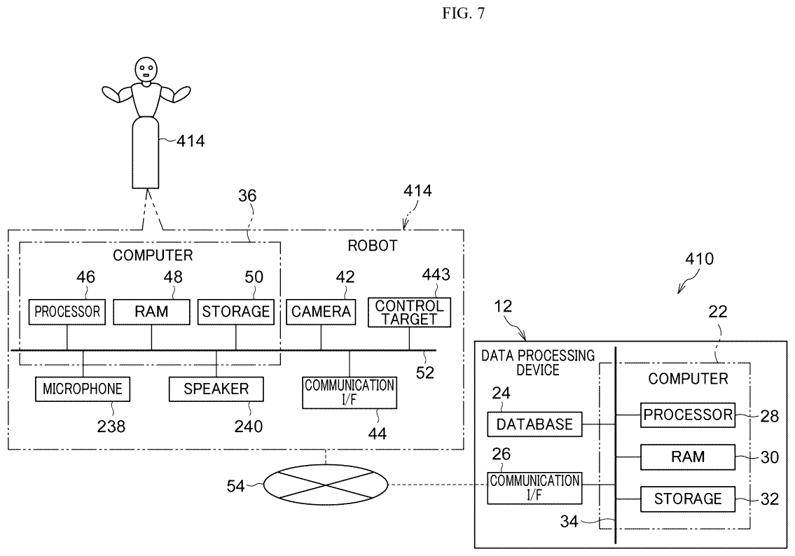

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

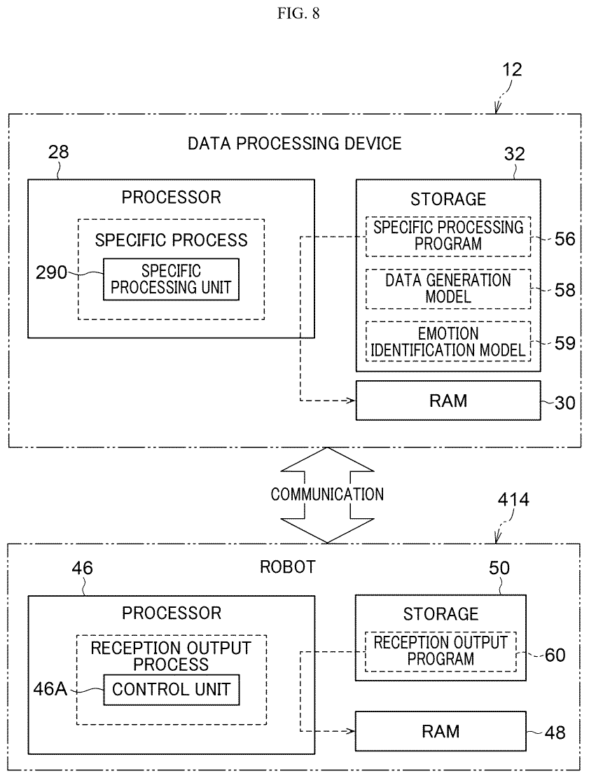

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

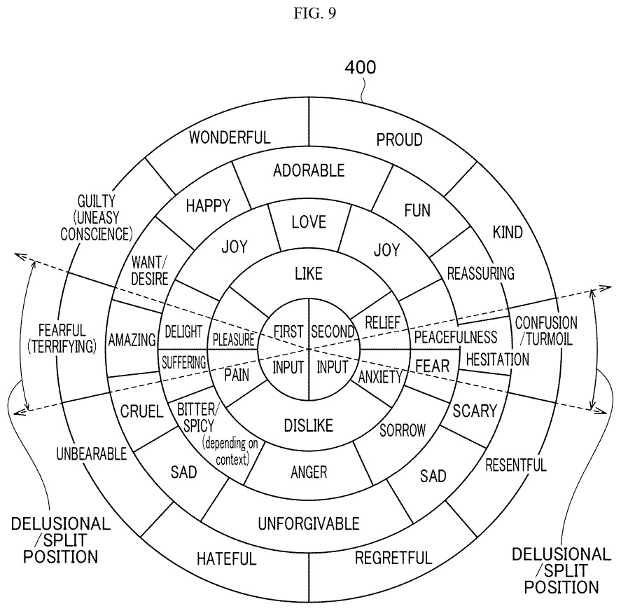

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

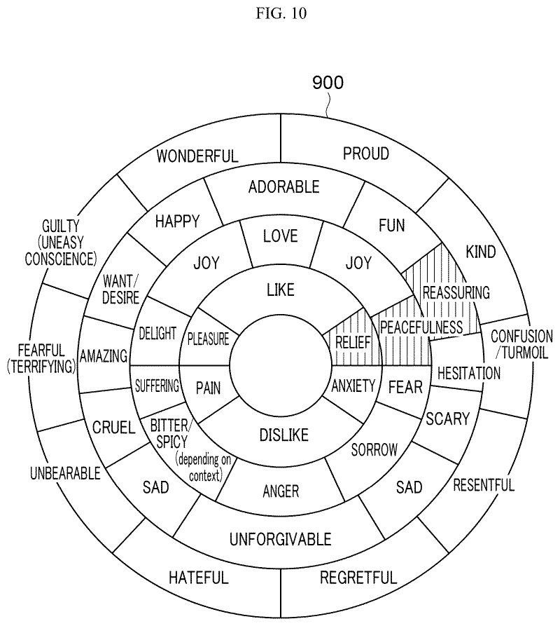

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire biological signals of a user, as digital signals, from a measurement apparatus via wireless communication,
transmit the acquired digital signals representing the biological signals to an information processing apparatus via a network,
receive, at the information processing apparatus, the digital signals representing the biological signals, and perform signal processing and machine learning processing on the digital signals to identify thought content of the user,
determine, based on the thought content of the user identified by the machine learning processing, information to be provided to the user or an operation to be executed, and generate a prompt sentence that instructs a generative AI model to perform a conversion for expressing the information or the operation in natural language,
control the generative AI model, executed as a program on at least one processing apparatus including the information processing apparatus or an external processing apparatus, to generate response information in natural language by using the prompt sentence as input, transmit the response information and command information corresponding to the thought content to a user terminal via the network, and
at the user terminal, control execution of an application program based on the command information, and present, via an output apparatus, at least one of a processing result of the application program or the response information to the user.

### (Supplementary 2)

The system according to supplementary 1,
wherein the processor is configured to
perform, in the information processing apparatus, data pre-processing for extracting temporal and spatial features of the biological signals in the machine learning processing, estimate a probability distribution corresponding to a plurality of thought states, determine the thought content representing a behavior request of the user based on the probability distribution, and dynamically change content of the prompt sentence according to a type of the determined thought content and a state of the user.

### (Supplementary 3)

The system according to supplementary 1,
wherein the processor is configured to
generate, in the information processing apparatus, the prompt sentence that instructs the generative AI model to generate natural language text in a plurality of languages or to perform translation between different languages according to the state and the thought content of the user estimated from the biological signals, and control the user terminal to present the response information in the plurality of languages simultaneously or sequentially.

### Application Example 1

### (Supplementary 1)

A system comprising a processor,
wherein the processor is configured to
measure a biological signal of a user and acquire the biological signal as time-series data, preprocess the time-series data and convert the time-series data into machine-learning input data by performing at least frequency-component extraction and feature generation on the time-series data,
classify, by using a statistical learning model, the machine-learning input data to determine thought information by identifying at least one of a thought content and an emotional state of the user,
determine, by referring to the thought information and profile information of the user, an information type and an information output format corresponding to the thought information, and generate prompt data including a prompt sentence for instructing a conversion of the thought information into natural language,
input the prompt data into a generative AI model and instruct the generative AI model to generate at least one of response information in natural language and content specification information,
acquire, on the basis of at least one of the content specification information and the response information, candidate content from at least one of an external information providing apparatus and an information storage apparatus, and determine personalized presentation content by selecting or composing the candidate content based on at least the thought information and history information of the user,
convert the personalized presentation content into output data, transmit the output data to a terminal device, and cause the terminal device to present the personalized presentation content as at least one of visual information and auditory information to the user, and
acquire, from the terminal device, feedback information including at least one of operation information and evaluation information of the user with respect to the personalized presentation content, store the feedback information in association with the thought information and the personalized presentation content, and accumulate the feedback information as learning data for updating at least one of the statistical learning model and a generation process of the prompt data.

### (Supplementary 2)

The system according to supplementary 1,
wherein the processor is configured to generate the prompt sentence so as to specify, based on the thought information, at least one of a type, a mood, a length, an output format, and a metadata item of the personalized presentation content to be generated, and to determine the personalized presentation content by analyzing content specification information in natural language obtained from the generative AI model as structured data in a machine-readable format.

### (Supplementary 3)

The system according to supplementary 1,
wherein the processor is configured to dynamically change a content of the prompt sentence in the generation process of the prompt data based on at least the thought information, the feedback information, time information, and usage-context information, and to update at least one of output conditions and prohibition conditions requested from the generative AI model so as to improve, over time, a selection accuracy of the personalized presentation content.

### Example 2

### (Supplementary 1)

A system comprising a processor,
wherein the processor is configured to
acquire, via wireless communication from a biological signal acquisition apparatus worn by a user, time-series digital signals based on brain activity of the user, and perform signal processing on the time-series digital signals to extract predetermined frequency-band components and calculate feature values,
identify, by executing inference processing using a machine learning model that receives the feature values as input, abstracted thought pattern information corresponding to a thought content of the user, generate structured data including the thought pattern information, and transmit the structured data to an external apparatus via a network,
construct, in accordance with the thought pattern information and a target output language, a prompt sentence to be input to a generative AI model for natural language generation,
transmit the prompt sentence to the generative AI model, obtain a natural language text from the generative AI model, and transmit the obtained natural language text to a user terminal via the network, and
cause, in the user terminal, the received natural language text to be presented by at least one of display output and audio output generated by a speech synthesis process.

### (Supplementary 2)

The system according to supplementary 1,
wherein the processor is configured to
generate the prompt sentence such that the prompt sentence includes content description information based on the thought pattern information, instruction information regarding a natural language style, output format, and target expression level, and translation instruction information for a plurality of target languages, and cause a plurality of natural language texts in the plurality of target languages, obtained from the generative AI model, to be output in association with the user terminal.

### (Supplementary 3)

The system according to supplementary 1,
wherein the processor is configured to
store, in a storage device, the thought pattern information identified by the machine learning model and the natural language text obtained from the generative AI model, and, based on accumulated history information, update at least one of configuration conditions of the prompt sentence and inference conditions of the machine learning model so as to adaptively optimize, for each user, a correspondence between thought patterns and natural language expressions.

### Application Example 2

### (Supplementary 1)

A system comprising a processor,
wherein the processor is configured to
acquire a biological signal of a user and obtain the biological signal as a digital signal, perform signal processing on the obtained digital signal to extract a feature amount and, on the basis of the feature amount, specify thought content of the user and estimate an emotional state of the user,
generate a prompt sentence, on the basis of the specified thought content and the estimated emotional state, the prompt sentence instructing a generative data generation apparatus to generate natural language, to translate or summarize natural language, or to generate control data for an operation apparatus,
input the prompt sentence into the generative data generation apparatus and obtain, from the generative data generation apparatus, a generation result as natural language data or as structured data,
generate audio data by using a speech synthesis apparatus on the basis of the obtained generation result and adjust an audio output characteristic of the audio data in accordance with the estimated emotional state, and
control an information processing apparatus or an operation apparatus in accordance with the obtained generation result or control data generated on the basis of the obtained generation result.

### (Supplementary 2)

The system according to supplementary 1,
wherein the processor is configured to
convert the natural language data obtained from the generative data generation apparatus into at least one of a search request for a search processing apparatus, a response sentence in dialog processing, and a control instruction for the operation apparatus, and to present a search result from the search processing apparatus or an operation state of the operation apparatus to the user by using the speech synthesis apparatus and a display apparatus.

### (Supplementary 3)

The system according to supplementary 1,
wherein the processor is configured to
add, to the prompt sentence, information for specifying an input language, an output language, and an output format, and to cause the generative data generation apparatus to perform at least one of translation between different languages, simultaneous output in a plurality of languages, and conversion into control-oriented structured data.

What is claimed is:

1. A system comprising:
circuitry configured to:
acquire a biological signal of a user as time-series data;
preprocess the time-series data by performing at least frequency-component extraction and feature generation to convert the time-series data into machine-learning input data;
classify the machine-learning input data using a statistical learning model to determine thought information by identifying at least one of a thought content and an emotional state of the user;
determine, by referring to the thought information and profile information of the user, an information type and an information output format corresponding to the thought information;
generate prompt data comprising a prompt sentence for instructing a conversion of the thought information into natural language;
input the prompt data to a generative artificial intelligence model to generate at least one of response information in natural language and content specification information;
acquire candidate content from at least one of an external information providing apparatus and an information storage apparatus based on at least one of the content specification information and the response information, and determine personalized presentation content by selecting or composing the candidate content based on at least the thought information and history information of the user; and
convert the personalized presentation content into output data and transmit the output data to a terminal device for presentation to the user as at least one of visual information and auditory information.

2. The system of claim 1, wherein the circuitry is configured to:
acquire feedback information comprising at least one of operation information and evaluation information of the user with respect to the personalized presentation content from the terminal device;
store the feedback information in association with the thought information and the personalized presentation content; and
accumulate the feedback information as learning data for updating at least one of the statistical learning model and a generation process of the prompt data.

3. The system of claim 2, wherein updating the statistical learning model based on the accumulated learning data comprises applying a supervised learning algorithm that uses labeled pairs of machine-learning input data and corresponding thought information as training examples.

4. The system of claim 3, wherein updating the generation process of the prompt data comprises adjusting a prompt template or a prompt parameter based on a reward signal derived from the evaluation information, such that prompt sentences associated with higher evaluation scores receive increased weights in subsequent prompt generation.

5. The system of claim 4, wherein the reward signal is computed as a function of at least one of a user satisfaction score, an engagement duration, and an accuracy of the response information as assessed by the evaluation information.

6. The system of claim 1, wherein frequency-component extraction applies at least one of a fast Fourier transform, a short-time Fourier transform, or a wavelet transform to the time-series data to compute spectral features within predefined frequency bands.

7. The system of claim 6, wherein the predefined frequency bands correspond to at least one of delta, theta, alpha, beta, and gamma brainwave frequency ranges, and wherein feature generation computes at least one of power spectral density, band power ratios, and temporal envelope features within the predefined frequency bands.

8. The system of claim 1, wherein the statistical learning model applies a convolutional neural network, a recurrent neural network, or a transformer-based architecture to the machine-learning input data to generate a probability distribution over a predefined set of thought content categories and emotional state categories.

9. The system of claim 8, wherein classifying the machine-learning input data comprises selecting as the thought information the thought content category and the emotional state category with the highest probabilities from the probability distribution generated by the statistical learning model.

10. The system of claim 1, wherein generating prompt data comprises serializing the thought information, the information type, the information output format, and profile information of the user into a structured prompt template that instructs the generative artificial intelligence model to generate response information constrained to the information type and formatted according to the information output format.

11. The system of claim 10, wherein the information output format specifies at least one of a language code, a content length constraint, a formality level, and a presentation modality for the response information.

12. The system of claim 1, wherein acquiring candidate content comprises transmitting a content query to the external information providing apparatus using at least one of a keyword derived from the content specification information and a semantic embedding vector derived from the response information, and receiving candidate content items matching the content query.

13. The system of claim 12, wherein determining personalized presentation content comprises ranking the candidate content items by a personalization score computed from a similarity between each candidate content item and the history information of the user, and selecting candidate content items with personalization scores above a predefined threshold.

14. The system of claim 1, wherein the biological signal comprises at least one of a brainwave signal, a cardiac activity signal, a muscle activity signal, or a skin conductance signal acquired by a sensor coupled to the user.

15. The system of claim 14, wherein preprocessing the time-series data further comprises applying at least one of a bandpass filter, a notch filter, and an artifact rejection algorithm to remove noise components from the time-series data prior to frequency-component extraction.

16. The system of claim 1, wherein the terminal device presents the output data as auditory information by driving an audio output device, and wherein the information output format specifies a speech synthesis parameter for converting the response information into an audio signal.

17. The system of claim 16, wherein the speech synthesis parameter specifies at least one of a speech rate, a pitch range, and a voice characteristic selected based on the emotional state of the user determined from the thought information.

18. A system comprising:
circuitry configured to:
acquire a biological signal of a user as time-series data and preprocess the time-series data by performing frequency-component extraction and feature generation to generate machine-learning input data;
classify the machine-learning input data using a statistical learning model to determine thought information identifying at least one of a thought content and an emotional state of the user;
generate prompt data comprising a prompt sentence encoding the thought information, an information type, and an information output format, and input the prompt data to a generative artificial intelligence model to generate response information;
acquire candidate content from at least one of an external information providing apparatus and an information storage apparatus and determine personalized presentation content based on the candidate content, the thought information, and history information of the user; and
acquire feedback information from a terminal device with respect to the personalized presentation content and accumulate the feedback information as learning data for updating at least one of the statistical learning model and a generation process of the prompt data.

19. The system of claim 18, wherein the circuitry is configured to apply a supervised learning algorithm that uses labeled pairs of machine-learning input data and corresponding thought information as training examples to update the statistical learning model, and to adjust a prompt template based on a reward signal derived from the feedback information to update the generation process of the prompt data.

20. A method performed by circuitry, the method comprising:
acquiring a biological signal of a user as time-series data;
preprocessing the time-series data by performing at least frequency-component extraction and feature generation to convert the time-series data into machine-learning input data;
classifying the machine-learning input data using a statistical learning model to determine thought information by identifying at least one of a thought content and an emotional state of the user;
generating prompt data comprising a prompt sentence encoding the thought information, an information type, and an information output format corresponding to the thought information;
inputting the prompt data to a generative artificial intelligence model to generate at least one of response information in natural language and content specification information;
acquiring candidate content from at least one of an external information providing apparatus and an information storage apparatus and determining personalized presentation content by selecting or composing the candidate content based on the thought information and history information of the user;
transmitting output data representing the personalized presentation content to a terminal device for presentation to the user; and
acquiring feedback information from the terminal device and accumulating the feedback information as learning data for updating at least one of the statistical learning model and a generation process of the prompt data.
